(19) United States
(12) Patent Application Publication
(10) Pub. No.: US 2023/0301584 A1
(43) Pub. Date: Sep. 28, 2023

(54) NANOSENSOR FOR REAL-TIME MONITORING OF WOUND HEALING, MANUFACTURING METHOD THEREOF, AND REAL-TIME MONITORING SYSTEM FOR WOUND HEALING USING SAME

(71) Applicants: Korea University Research and Business Foundation, Seoul (KR); City University of Hong Kong, Hong Kong (CN)

(72) Inventors: Woo young JANG, Seoul (KR); Jangsun HWANG, Seoul (KR); Chenjie XU, Hong Kong (CN)

(73) Assignees: Korea University Research and Business Foundation, Seoul (KR); City University of Hong Kong, Hong Kong (CN)

(21) Appl. No.: 18/057,736

(22) Filed: Nov. 21, 2022

Related U.S. Application Data
(60) Provisional application No. 63/303,121, filed on Jan. 26, 2022.

(51) Int. Cl. A61B 5/00

(52) U.S. Cl. CPC A61B 5/445; A61B 5/0071; A61B 2562/0285; A61B 2562/12

(57) ABSTRACT

The present disclosure relates to a nanosensor for real-time monitoring of wound healing, a manufacturing method thereof, and a real-time monitoring system for wound healing using the same. A nanosensor according to example embodiments includes a biomarker for detecting mRNA and a reference gene on gold nanoparticles, thereby providing an objective indicator through monitoring of wound healing, enabling monitoring of wound healing through direct monitoring using real-time fluorescence by including a fluorescent marker in a nanoflare, and enabling evaluation of a whole wound healing process in normal and patient groups (diabetes) through real-time monitoring. In addition, the nanosensor according to the example embodiments has advantages of shortening synthesis time and improving efficiency by 30% through integration of a novel synthesis method (freezing method) rather than an existing synthesis method (salt aging).

### CROSS-REFERENCE TO RELATED APPLICATIONS

This application claims priority to and the benefit of U.S. Provisional Pat. Application No. 63/303,121 filed on Jan. 26, 2022, in the United States Patent and Trademark Office, the entire disclosure of which is incorporated herein by reference.

### INCORPORATION-BY-REFERENCE OF MATERIAL SUBMITTED BY U.S.P.T.O. EFS-WEB

This application contains a Sequence Listing, which is being submitted in computer readable form via the United States Patent and Trademark Office Patent Center and which is hereby incorporated by reference in its entirety for all purposes. The XML file submitted herewith, which is named as “NewApp_0181960002_SequenceListing” and is created on Nov. 21, 2022, contains 80,532 bytes file.

### BACKGROUND

### 1. Field of the Invention

The present disclosure relates to a nanosensor for real-time monitoring of wound healing, a manufacturing method thereof, and a real-time monitoring system for wound healing using the same.

### 2. Description of the Related Art

Wound management is to control the intrinsic ability of tissues for regeneration of damaged or lost skin tissues. Wound healing is associated with spatial and temporal synchronization of various stages including homeostasis/coagulation, inflammation, proliferation/re-epithelialization, and remodeling. Wounds that fail to proceed with orderly and timely care for establishing anatomical and functional integrity are considered as abnormal wounds. Diabetes and chronic infections are particularly common causes of improper wound healing. A successful wound management strategy requires a timely and accurate assessment on the wound condition for effective clinical decision-making. Currently, wound management is carried out by simple visual observation of wounds or methods that are time consuming and destructive such as quantitative polymerase chain reaction (qPCR) to investigate specific metabolites or biomarkers in wound fluid.

Sensory nanomaterials, or nanosensors, rely on the intrinsic physicochemical properties of nanomaterials to enable faster, cheaper, more sensitive, and accurate medical diagnosis. Depending on properties of the nanomaterial, electrochemical, optical, or piezoelectric methods may be fundamentally used as a detection method.

Although fields such as diagnosis and prognosis of cancer and blood glucose monitoring have been widely studied, potential functions thereof in wound evaluation have yet been studied widely. Several existing studies target specific metabolites and biomarkers in wound fluid, focusing on a single stage of the wound healing process. However, as mentioned above, wound healing includes several stages with unique biomarkers involved therein. The overall process of the wound healing may only be revealed by continuous, collective analysis on multiple biomarkers at different stages of wound healing, and the limitations of existing studies may be unveiled in that invasive methods are applied.

Therefore, it is necessary to study on a method that involves diffusion through the skin layer using an optical sensor for evaluation on wound healing, targets and quantifies mRNA biomarkers of target cells, and enables objective observation of wound healing in a non-invasive manner.

### Prior Art Document

### [Non-Patent Document]

J. Am. Chem. Soc. 2017, 139, 9471-9474 (published on Jun. 29, 2017)

### SUMMARY

An object of the present disclosure is to provide a nanosensor which enables detection of the wound healing progress and objective evaluation through direct monitoring of the wound using real-time fluorescence for inflammatory response, cell proliferation, and/or angiogenesis (inflammation) during the wound healing process, by including a nanoflare for mRNA detection. The present disclosure also provides a manufacturing method thereof, a real-time monitoring system for wound healing using same, and a monitoring method.

Example embodiments of the present disclosure provide a nanosensor for monitoring wound healing, including a nanoflare consisting of a core part including gold nanoparticles, and a flare part including a recognition sequence and a flare sequence, wherein the recognition sequence complementarily binds to a target gene, the flare sequence complementarily binds to the recognition sequence, and the flare part is formed on the surface of the core part.

In addition, example embodiments of the present disclosure provide a method of manufacturing the nanosensor for monitoring of wound healing processes described above, including preparing a flare part by mixing a recognition sequence and a flare sequence, and storing a mixture obtained by mixing the prepared flare part with gold nanoparticles at a temperature of -30 to -10° C. for 1 hour to 3 hours and reacting the mixture with a salt to form a nanoflare.

In addition, example embodiments of the present disclosure provide a wound healing monitoring system including the nanosensor described above.

In addition, example embodiments of the present disclosure provide a wound healing monitoring method including applying the above-described nanosensor to a wound site, and measuring a degree of wound healing through in vivo fluorescence imaging by a fluorescent dye included in the flare sequence, as a recognition sequence in the applied nanosensor binds to a target gene in the wound site.

A nanosensor according to example embodiments of the present disclosure includes a flare part capable of binding to a biomarker for mRNA detection and a reference gene on gold nanoparticles to provide an objective indicator through wound healing monitoring, enables monitoring of wound healing via direct monitoring using real-time fluorescence by including a fluorescent marker in the nanoflare as well as evaluation on the whole process of wound healing through real-time monitoring in a normal group and a patient group (diabetes).

In addition, the nanosensor according to the example embodiments has advantages of shortening synthesis time and improving efficiency by 30% through integration of a novel synthesis method (freezing method) rather than an existing synthesis method (salt aging).

### BRIEF DESCRIPTION OF THE DRAWINGS

FIG. 1 shows results of screening biomarkers for fibroblasts (NDF), keratinocytes (HaCaT), and endothelial cells (HUVEC): representing cellular expression of 30 potential RNA biomarkers in (A) NDF; (B) HaCaT; and (C) HUVECs, as well as cellular expression of 10 identified (D) NDF biomarkers; (E) HaCaT biomarkers; and (F) HUVEC biomarkers in NDF, HaCaT, and HUVEC, respectively.

FIG. 2 is a schematic diagram and an experimental result showing the optimization of R:F ratio for R-F hybridization.

FIG. 3 shows experimental results on features of a nanoflare (NF): (A) is a graph showing the change in the fluorescence signal of the nanoflare prepared by a conventional method and a manufacturing method according to an example embodiment of the present disclosure, (B) is absorbance spectra of gold nanoparticles and the nanoflare, (C) is a result of measuring hydrodynamic diameters of gold nanoparticles and the nanoflare, and (D) is a graph showing the zeta potential of gold nanoparticles and the nanoflare (n=3, values are mean ± s.d.).

FIG. 4 shows schematic diagrams of selected recognition sequence (R) and flare sequence (F): (A) is a possible secondary structure of the R-F duplex for a nanoflare targeting FSP1, (B) is a possible secondary structure of the R-F duplex for a nanoflare targeting KRT14, (C) is a possible secondary structure of R-F duplex for a nanoflare targeting PECAM1, and (D) is a possible secondary structure of R-F duplex for a nanoflare targeting GAPDH.

FIG. 5 shows experimental data obtained by measuring the change in the fluorescence signal of a nanoflare in accordance with the nucleotide length: (A) is a graph quantifying the change in the fluorescence signal in accordance with the nucleotide length of FSP1-NF, and (B) is a graph quantifying the changes in the fluorescence signal over time.

FIGS. 6A to 6I show experimental results on the sensitivity of a nanoflare according to an example embodiment of the present disclosure: (A) is a schematic diagram of the binding between the nanoflare and a target sequence, and graphs showing fluorescence restoration of (B)FSP1-NF, (D) KRT14-NF, (F) PECAM1-NF, and (H) GAPDH-NF over time after mixing a concentration-fixed target sequence with a mismatched sequence (concentration of the target sequence or mismatched target sequence is 2 µM, concentration of the nanoflare is OD 0.4, black line = PBS); graphs showing fluorescence restoration of (C) FSP1-NF, (E) KRT14-NF, (G) PECAM1-NF, and (I) GAPDH-NF after mixing with various concentrations of target sequence and mismatched sequence (concentration of the nanoflare is OD 0.4, black line: mismatched sequence=2 µM, the incubation time is 20 min, n=2, values are mean ± sd, FAM excitation emission=495-520 nm, Cy3=550-570 nm, Cy5=633-647 nm).

FIG. 7 shows experimental results on the concentration of the flare sequence of a nanoflare according to an example embodiment of the present disclosure: graphs showing the fluorescence restoration of (A) FSP1-NF, (B) KRT14-NF, (C) PECAM1-NF, and (D) GAPDH-NF at various concentrations of the target sequence (n=2, values are the mean ± s.d.).

FIG. 8 shows experimental results on concentration optimization of a nanoflare according to an example embodiment of the present disclosure: graphs showing the working concentration of (A) FSP1-NF, (B) KRT14-NF, (C) PECAM1-NF, and (D) GAPDH-NF to be used for cell experiments (n=3, 5x10⁴/mL cells were labeled with NF within 24 hours, **p <0.01, ***p <0.001, ****p <0.0001, and ns=not significant, n=3, values are the mean ± s.d.).

FIG. 9 shows experimental results on the cytotoxicity of a nanoflare according to an example embodiment of the present disclosure: graphs showing the cytotoxicity of (A) FSP1-NF against NDF, (B) KRT14-NF against HaCaT, (C) PECAM1-NF against HUVEC, and (D) GAPDH-NF against HUVEC.

FIGS. 10A to 10I show results of target gene expression evaluation of cells for a nanoflare according to an example embodiment of the present disclosure.

FIGS. 11A to 11I are graphs quantifying the expression of a target gene in cells by a nanoflare according to an example embodiment of the present disclosure.

FIG. 12 shows images of confocal fluorescence of NDF labeled with FSP1-NF at different time points by using a nanoflare according to an example embodiment of the present disclosure.

FIGS. 13A to 13D show results of evaluation and identification of target gene expression under stimulation by a nanoflare (NF) and qPCR according to an example embodiment of the present disclosure: (A) is a schematic diagram thereof; (B) is an expression result of FSP1 in NDF treated with 10 growth factors, (C) is a result of KRT14 expression in HaCaT treated with 10 growth factors, and (D) is a result of expression of PECAM1 in HUVEC treated with 10 growth factors, wherein signals of FSP1-NF, KRT14-NF, and PECAM1-NF are normalized using the signals of GAPDH-NF, and the values are mean ± s.d.

FIG. 14 shows fluorescence images of NDF treated with a nanoflare according to an example embodiment of the present disclosure and 10 growth factors.

FIG. 15 shows fluorescence images of HaCaT treated with a nanoflare according to an example embodiment of the present disclosure and 10 growth factors.

FIG. 16 shows fluorescence images of HUVEC treated with a nanoflare according to an example embodiment of the present disclosure and 10 growth factors.

FIG. 17 shows confocal fluorescence images of 3D spheroid and 2D cell culture using a nanoflare according to an example embodiment of the present disclosure: (A) is a schematic diagram, (B) is results of 2D and 3D co-culture of NDF and HaCaT using the nanoflare (white scale bar is 50 µm), and (C) top images represent KC in the presence of the nanoflare, middle images represent DP in the presence of the nanoflare, and the botteom images represent co-culture of KC and DP in the presence of the nanoflare (scale bar is 200 µm), wherein the concentrations of FSP1-NF, KRT14-NF, and GAPDH-NF are O.D 0.1, 0.0125, and 0.0125, respectively.

FIGS. 18A to 18E show results of monitoring the changes in cellular genes of the 3D spheroid using a nanoflare according to an example embodiment of the present disclosure.

FIGS. 19A to 19H showresults of monitoring wound healing by a nanoflare of an example embodiment of the present disclosure applied topically to normal and diabetic mice: (A) is a schematic diagram of an experimental configuration; (B) shows the blood glucose level, (C) is the wound site, and (D) shows the change in body weight of normal and diabetic mice through the experiment; (E) is a group of normal mice, (F) is a group of diabetic mice (the signal is GAPDH which is a reference gene, normalized to the mean signal using SEM); (G) is a comparison of the days when the highest fluorescence signal of a nanoflare was observed for each biomarker in two wound healing models in E and F (*p <0.05, ** p <0.01, *** p <0.001, **** p <0.0001, ns=not significant, n=6, duplicates); (H) is the fluorescence wound healing index (FWI) for the normal and diabetic groups (a mean value of the highest fluorescence signal for nanoflare is normalized to GAPDH signal after day 10 in both groups, n=11, values are mean ± s.d.).

FIGS. 20A to 20E show results of tracking a wound healing process in normal and diabetic wound healing models using a nanoflare according to an example embodiment of the present disclosure.

FIG. 21 shows images of H&E staining under an optical microscope, illustrating mouse test results using a nanoflare according to an example embodiment of the present disclosure: (A) uninjured state; (B) wound after two (2) days; (C) wound after four (4) days; (D) wound after seven (7) days; (E) wound after ten (10) days; and (F) a schematic diagram of an experiment conducted by forming a wound on the back of a mouse with a 4 mm punch.

FIG. 22 shows (A) a graph illustrating a wound healing process of a normal mouse; and (B) a graph analyzing wound healing based on the nanoflare signal.

FIG. 23 shows images illustrating the wound healing process of diabetic foot ulcer: (A) a delayed proliferation stage; (B) proliferating but lack of an epithelialization stage; (C) initiation of re-epithelialization; and (D) successful re-epithelialization.

### DETAILED DESCRIPTION

Hereinafter, the present disclosure will be described in detail.

An example embodiment of the present disclosure provides a nanosensor for monitoring wound healing, including a nanoflare including a core part including gold nanoparticles, and a flare part including a recognition sequence and a flare sequence, wherein the recognition sequence complementarily binds to a target gene, the flare sequence complementarily binds to the recognition sequence, and the flare part is formed on the surface of the core part.

The nanosensor according to an example embodiment of the present disclosure provides an objective indicator by monitoring of wound healing, enabling direct monitoring of differentiation and growth of target cells or mRNA by being used as a patch, a band, and a scaffold. In addition, the nanosensor may replace a quantitative analysis method of genes using qPCR, and direct monitoring of diseases by gene expression is possible as well. Further, it is also possible to present direct evidence for cancer cell growth, drug-induced reduction, and drug-induced organ damage.

The nanosensor may measure a degree of wound healing as the flare part binds to the target gene. Specifically, the nanosensor may measure the degree of wound healing as the recognition sequence of the flare part complementary binds to the target gene. More specifically, the nanosensor may measure the degree of wound healing as the fluorescence signal changes in accordance with the concentration of the target gene.

The gold nanoparticles may have a structure in which citrate is bound to the surface of gold nanoparticles.

The recognition sequence may complementarily bind to the target gene, and the recognition sequence may include a base sequence capable of recognizing the target gene. Specifically, the recognition sequence may include a thiol-modified sequence at the 3′ end of the base sequence recognizing the target gene.

The target gene may include one or more selected from the group consisting of Platelet And Endothelial Cell Adhesion Molecule 1 (PECAM1), Fibroblast-specific protein 1 (FSP1), Keratin 14 (KRT14), and Glyceraldehyde 3-phosphate dehydrogenase (GAPDH). Specifically, the base sequence recognizing the target gene may be one or more of base sequences represented by SEQ ID NO: 1, SEQ ID NO: 2, SEQ ID NO: 9, SEQ ID NO: 10, SEQ ID NO: 13, SEQ ID NO: 15, and SEQ ID NO: 16. More specifically, the base sequence recognizing the target gene may include one of base sequences represented by SEQ ID NO: 1, SEQ ID NO: 2, SEQ ID NO: 9, SEQ ID NO: 10, and SEQ ID NO: 13; and a base sequence represented by SEQ ID NO: 15 or SEQ ID NO: 16. The base sequence represented by SEQ ID NO: 15 or SEQ ID NO: 16 is a base sequence recognizing GAPDH, enabling quantitative evaluation of the expression of the target gene as a reference gene.

The flare sequence may consist of 14 to 18 nucleotides.

The flare sequence may include one or more of base sequences represented by SEQ ID NO: 3 to SEQ ID NO: 8, SEQ ID NO: 11, SEQ ID NO: 12, SEQ ID NO: 14, SEQ ID NO: 17, and SEQ ID NO: 18. Specifically, the flare sequence may include one base sequence represented by SEQ ID NO: 3 to SEQ ID NO: 8, SEQ ID NO: 11, SEQ ID NO: 12, or SEQ ID NO: 14; and a base sequence represented by SEQ ID NO: 17 or SEQ ID NO: 18.

The flare sequence may include a fluorescent dye at the 5′ end. Specifically, the fluorescent dye may include at least one selected from the group consisting of Cy3, Cy5, FAM, FITC, Cy5.5, RITC, TAMRA, and Texas red.

A binding force between the recognition sequence and the flare sequence may be 15 kcal/mol or more, and the binding force between the recognition sequence and the target gene may be 60 kcal/mol or less. Specifically, the binding force between the recognition sequence and the flare sequence may be 18 kcal/mol or more, and the binding force between the recognition sequence and the target gene may be 55 kcal/mol or less. Fluorescence efficiency may be the most excellent when exhibiting such a binding force.

In addition, an example embodiment of the present disclosure provides a manufacturing method of the nanosensor for monitoring wound healing described above, including preparing a flare part by mixing a recognition sequence and a flare sequence; and storing a mixture in which the prepared flare part mixed with gold nanoparticles at a temperature of -30 to -10° C. for 1 hour to 3 hours and reacting the mixture with a salt to form a nanoflare.

The flare part preparation may be performed by mixing the recognition sequence and the flare sequence in a certain ratio and annealing the mixture at a temperature from 60-120° C. to room temperature (20-25° C.) for 1 to 2 hours. In this case, the recognition sequence and the flare sequence may react to form a duplex.

In the flare part preparation, the recognition sequence and the flare sequence may be mixed in a molar ratio of 1:1 to 10:1, 1:1 to 5:1, or 3:1 to 5:1.

In the flare part preparation, the recognition sequence may be activated after formation of the duplex by a reaction between the recognition sequence and the flare sequence. Specifically, the recognition sequence may be activated by thiol modification. More specifically, the activation may be performed by treating at least one selected from the group consisting of tris(2-carboxyethyl)phosphine (TCEP), dithiothreitol (DTT), and mercaptoethanol.

The flare part preparation may involve addition of the flare sequence in which a fluorescent dye is bound to a terminal end of the flare sequence by mixing the flare sequence and the fluorescent dye. Specifically, the fluorescent dye may include at least one selected from the group consisting of Cy3, Cy5, FAM, FITC, Cy5.5, RITC, TAMRA, and Texas red.

In forming the nanoflare, a salt may be added to the mixture and reacted for 1 hour to 24 hours until the final salt concentration reaches 0.2 to 0.5 M or 0.2 to 0.4 M. Specifically, in the nanoflare formation, the salt may be added at intervals of 20 to 40 minutes until the salt concentration reaches the target concentration. The salt may be sodium chloride.

In forming the nanoflare, the prepared flare part and the gold nanoparticles may be mixed in a volume ratio of 1:3 to 10. Specifically, in forming the nanoflare, the prepared flare part and the gold nanoparticles may be mixed in a volume ratio of 1:5 to 10.

In the nanoflare formation, sonication may be additionally performed in the mixture. The sonication conditions may be to sonicate for 10 seconds to 60 seconds at intensity of 30 to 90 kHz, 35 to 85 kHz, or 37 to 80 kHz.

The method may further include, after the forming of the nanoflare, isolating the reaction mixture and then storing it in 0.01% to 0.5% PBST (Tween-20 in Phosphate-buffered saline solution) solution. In the case of storing in the buffered solution as described above, there are advantages of preventing aggregation of nanoparticles, providing a base useful for binding to a recognition site and mRNA, and preventing damage during cell and tissue processing.

Additionally, after the nanoflare formation, separation of the reacted mixture by centrifugation may further be included. The centrifugation may be repeated three or more times at 10,000 to 15,000 g for 10 to 60 minutes and at 10,000 to 20,000 rpm for 10 to 60 minutes.

In addition, an example embodiment of the present disclosure provides a wound healing monitoring system including the nanosensor described above.

The wound healing monitoring system may measure a degree of wound healing through in vivo fluorescence imaging by the fluorescent dye contained in the flare sequence, as the target gene in the wound site binds with the recognition sequence in the nanosensor after application of the nanosensor to the wound site.

In addition, an example embodiment of the present disclosure provides a wound healing monitoring method including applying the above-described nanosensor to a wound site; and measuring a degree of wound healing through in vivo fluorescence imaging by the fluorescent dye contained in the flare sequence as the recognition sequence in the applied nanosensor binds to a target gene in the wound site.

The wound healing monitoring method using the nanosensor according to an example embodiment of the present disclosure enables monitoring of normal and abnormal wound healing, thereby providing an appropriate treatment method by grasping real-time drug response and patient status.

The application of the nanosensor to the wound site may involve application of a mixture of an emulsion solution and the nanosensor. The emulsion solution may be a mixture of a moisturizing cream (Aquaphor) and a buffer solution in a weight ratio of 5-7:3-5.

The measuring of the degree of wound healing may include measuring an intensity of fluorescence of the fluorescent dye included in flare sequence. Specifically, the measuring of the degree of wound healing may include measuring the intensity of fluorescence of the fluorescent dye included in the nanoflare using an IVIS spectrum device.

The fluorescent dye may include at least one selected from the group consisting of Cy3, Cy5, FAM, FITC, Cy5.5, RITC, TAMRA, and Texas red.

In addition, in the measuring of the degree of wound healing, the base sequence represented by SEQ ID NO: 7 or SEQ ID NO: 8 may be a base sequence recognizing GAPDH, enabling quantitative evaluation on the target gene expression as a reference gene.

Specifically, in the measuring of the degree of wound healing, the degree of wound healing may be an inflammatory reaction stage if the wound healing index of the target gene of the nanosensor is 1 or more and 1 to 3 after day 2, day 2 to 5, or day 2 to 4. Specifically, the target gene may be PECAM1.

In addition, in the measuring of the degree of wound healing, the degree of wound healing may be a proliferation & re-epithelialization stage if the wound healing index of the target gene of the nanosensor is less than 1 after day 5 or 7. Specifically, the target gene is PECAM1.

In the measuring of the degree of wound healing, the degree of wound healing may be a proliferation & re-epithelialization stage if the wound healing index of the target gene of the nanosensor is 1 or more after day 5 or 7. Specifically, the target gene may be one or more selected from the group consisting of FSP1 and KRT14.

Hereinafter, the present disclosure will be described in more detail through examples. The examples are merely for illustrating the present disclosure in more detail, and it will be apparent to those skilled in the art that the scope of the present disclosure is not limited by the examples according to the gist of the present disclosure.

### Materials and Methods

Streptozotocin and growth factors (EGF, FGF2, TGFβ1, KGF, IGF, TNF-α, IL-6, PDGF, VEGF, and GM-CSF) were purchased from Sigma Aldrich (USA). PureLink™ RNA mini kit, tris(2-carboxyethyl)phosphine (TCEP), and Matrigel were purchased from Thermo Fisher Scientific. 13 nm citrate-capped GNPs were used. All DNA oligos were purchased from Integrated DNA Technologies equipped with HPLC purification. Other chemicals and agents were all purchased from Sigma Aldrich (USA), except for one specifically mentioned.

Cell maintenance: NDFs, Human keratinocytes (HaCaT), HUVEC were purchased from Cell Research Corporation (Singapore) and used between passages 3 and 10. HUVEC were maintained with Endothelial Cell Growth Medium-2 Bullet Kit (Lonza). All other cells were maintained with DMEM. All the media were supplemented with 1% antibiotics (penicillin 100 U/ml and streptomycin 100 µg/ml) and heat inactivated 10% fetal bovine serum (FBS) in 5% CO2 at 37° C.

qPCR analysis for selection of target sequences: Cells (NDF, HaCaT and HUVEC) were seeded on the 6-well plate without any stimulation at a density of 2×104 and cultured to reach the confluency of 80%. Cells were then harvested for the extraction of total RNA using PureLink™ RNA Mini Kit (Thermo Fisher Scientific). 1 µg RNA was reverse transcribed using qScript cDNA SuperMix (Quanta BioSciences). Real-time qPCR was performed on the cDNA with Light-Cycler®480 SYBR Green I Master on a CFX96 Touch System (Bio-Rad). Primers were purchased from Integrated DNA Technologies. mRNA expression was normalized to the GAPDH expression using the 2-ΔΔCt method. Each experiment was performed three times in duplicate. For the growth factor stimulation study, cells were firstly starved for 24 hrs in DMEM with 1% FBS before the addition of growth factors for 2 days.

NFs Design and Synthesis: Table 2 lists the thiol-modified recognition sequences (termed as R) and the flare sequences (termed as F) that were modified with fluorescent dyes at 5′ end. Recognition sequences are complementary to the target sequences. To optimize sensitivity and stability of NFs, we set the predicted free energy change of all R-F duplexes was >-20 kcal/mol and R-T (Recognition-Target) was <-60 kcal/mol Table 3).

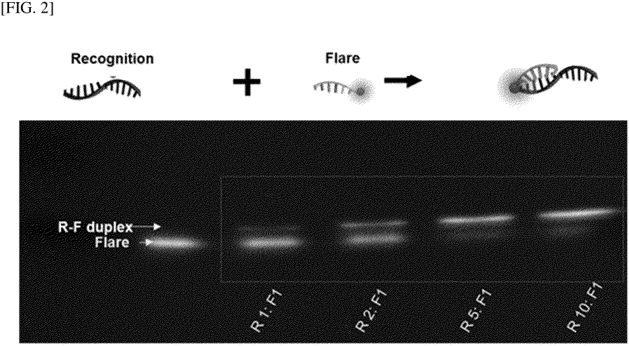

Recognition strands (20 µL, 100 µM) and flare strands (10 µL, 100 µM) were annealed from 95° C. to room temperature (RT) over 1 hr in different molar ratios (1:1, 2:1, 5:1, and 10:1, v/v) (FIG. 2). The resulted R-F duplexes were treated with 50 mM TCEP at RT for 1 hr to activate the thiol-modified recognition strand before being purified with Micro Bio-Spin 6 Columns (Bio-Rad). The purified R-T duplexes were added to 13 nm citrate-capped GNPs (1 mL, 5 nM). Next, the mixture was stored at -20° C. for 2 hrs, followed by adding 50 µL of 1 M NaCl every 30 mins until the final NaCl concentration reached 0.3 M. (26) The solution was sonicated for 30 s after each addition of NaCl. After stirred overnight, functionalized GNPs or NFs were derived after centrifugation at 14,000 g for 30 mins. NFs were washed twice with 0.05% Tween-20 in PBS. Their concentrations were determined by the absorbance at 520 nm (Nanodrop 2000, Thermo Fisher Scientific). Hydrodynamic size and zeta potential of SNAs were measured using a dynamic light-scattering system (Zetasizer, Malvern, UK).

Cytotoxicity test: HaCaT, NDFs and HUVEC were cultured in 96 well plate at the confluency at 80% and then treated with different concentrations of particles (O.D = maximum absorbance at 520 nm: value of 0, 0.05, 0.15, 0.25, 0.4 for both GNPs and NFs) for 24 hrs. Next, 100 µL of 1:20 diluted CCK-8 solution (CCK-8: DMEM, v/v) was added to each well and incubated for 4 hrs. The percentage of live and dead cells was spectrophotometrically analyzed at 450 nm.

Hybridization of NFs with free target strand: 50 µL of NFs at the O.D of 0.4 were mixed with target strands at 37° C. for 10 mins in the 96-well plate. The fluorescence intensities were examined at 520 nm for FSP1-FAM NF, 570 nm for KRT14-Cy3 NF, 570 nm for PECAM1-Cy3 NF, and 670 nm for GAPDH-Cy5 NF using Synergy HT Microplate Reader (BioTek). The concentrations of target strand were ranged from 0 to 10 µM to evaluate the sensitivity of NFs. Each measurement was repeated three times in duplicate.

Hybridization of NFs with target RNA in the cellular RNA extract: RNA extraction from 2D cultured cells was carried using PureLink™ RNA Mini Kit (Thermo Fisher Scientific). Then NFs were mixed with the total cellular RNA extract at RT for 30 mins. The fluorescence intensities of the mixture were examined using Synergy HT Microplate Reader (BioTek). Each measurement was repeated three times in duplicate.

NFs for quantifying cellular expression of target genes: Cells on 48-well plate were starved for 24 hrs in DMEM with 1% FBS. Later FGF2/TGFβ1, EGF/TGFβ1, and VEGF/TGFβ1 were added to the media for NDF, HaCaTs and HUVEC respectively. 2 days later, the cells were washed with PBS and fed with 5% FBS medium containing NFs (the concentrations were O.D 0.125 and 0.1). 16 hrs later, cells were stained with Hoechst 33342 and subjected to confocal imaging (LSCM, Carl Zeiss). Additionally, total RNA was extracted from the same experimental groups, and then treated with NFs before the measurement of fluorescence intensity. qPCR was also performed with the extracted RNA to compare with results from NF quantification. The comparison of target gene expression in NDF, HaCaT, and HUVEC were carried in a similar way but without stimulation.

NFs for identifying the potent growth factors: Cells were cultured in the 48-well plate and starved for 24 hrs before individual growth factor was added (EGF: 40 ng/mL, FGF2: 40 ng/mL, TGFβ1: 40 ng/mL, KGF: 40 ng/mL, IGF:50 ng/mL, TNF-α: 40 ng/mL, IL-6: 40 ng/mL, PDGF: 8 ng/mL, VEGF: 20 ng/mL, GM-CSF: 20 ng/mL, in 1% FBS/DMEM) and incubated for 48 hrs. Next, DMEM containing O.D 0.1 FSP1-NF, O.D 0.0125 KRT14-NF, PECAM1-NF and GAPDH-NF were added for another 16 hrs. After washing, 1 mL of Hoechst 33342 working solution was added for 30 mins to stain the cell nuclei. Cells were washed with PBS two times and imaged under confocal microscope (Carl Zeiss). The laser intensity and exposure were kept constant among experiments, which were performed in RT with minimized background light.

Spheroid formation and NF penetration assay: Immortalized normal human keratinocytes (KCs, N/TERT-1, courtesy of Prof. JG Rheinwald) and human primary hair follicle dermal papilla cells (DPCs, Adult, Cell Application Inc.) were used to generate the 3D spheroid cultures. KCs and DPCs were grown in Keratinocyte serum-free media (KSFM) and DMEM supplemented with 10% fetal bovine serum (Hyclone) and 0.14 mg/mL bone pituitary extract (Gibco), respectively. Single cell suspensions were seeded into low attachment 96-well plates (Corning) and incubated at 37° C. for spheroid formation.

To generate spheroids, 2000 KCs, 2000 DPCs and 2000 KCs + 2000 DPCs were seeded into each well to generate KC, DP, and the KCDP spheres. The spheroids were collected at day 3 post-seeding and incubated in 100 µL of 1% FBS DMEM containing a mixture of KRT14-NF, FSP1-NF, and GAPDH-NF (2 - 5 µL/mL, at the concentration of OD 0.1 - 0.125) for 16 hrs. The spheroids were then washed once with PBS and stained with Hoechst 33342 to stain the nuclei. Cy3, 6-FAM, and Cy5 filters were used to visualize the signals of KRT14-NF, FSP1-NF, and GAPDH-NF, respectively, on the Olympus FV3000RS inverted confocal microscope. For all samples, Z-stacks were collected and processed using FIJI software to obtain the sum slices projection images.

Small Interfering RNA (siRNA) Transfection on 3D spheroid: To generate spheroids for the use in the NFs sensitivity assay, 2000 NDF, 2000 HaCaT and 2000 NDF + 000 HaCaT were seeded per well on day 0. On day1 post-seeding, the spheroids were transfected with 10 µM of TGFBR1 siRNA duplexes (Thermofisher Scientific) using Lipofectamine® 2000 (ThermoFisher Scientific), according to manufacturer’s protocol, and incubated at 37° C. for 24 hrs. The transfection media was then replaced with 100 µL of 1% FBS DMEM with or without TGFβ1 (10 ng/mL) and incubated at 37° for 24 hrs. Following these treatments, the spheres were incubated in 100 µL of 1% FBS DMEM containing either KRT14-NF and GAPDH-NF (HaCaT spheres) or FSP1-NF and GAPDH-NF (NDF spheres) for another 16 hrs. The spheroids were then washed once with PBS and stained with Hoechst 33342. Cy3 filter was used to visualize the signals of KRT14-NF and FSP1-NF, while GAPDH-NF signal was detected using Cy5 filter on the Olympus FV3000RS inverted confocal microscope. For all samples, Z-stacks were collected and processed using FIJI software to obtain the sum slices projection images.

Monitoring NFs signal on normal Mouse model: Briefly, C57BL/6 were anaesthetized using alfaxalone (30~60mg/kg) before generating wounds on the mouse back. Alfaxalone was diluted 1:10 with PBS and injected intramuscularly at 0.1 mL per mouse. The wounds were created using a 4 mm biopsy punch and each mouse has 4 spots. When removing the skin, care was taken to ensure complete removal of the attached connective tissue, and the flap exposure.

To remove variability from individual wounds as a result of anatomical location, the topical applications (GAPDH-Cy5, PECAM-Cy3, and KRT14-Cy3 and FSP1-Cy3) were assigned to 4 wounded spots for experimental group. The order of topical application was rotated clockwise. In the case of the control group, the number of wounded spots was the same for uniformity of the experiment, but only GAPDH and GNPs were treated. Moisture cream (Aquaphor) and PBS were mixed at a ratio of 6:4 to make a formulation suitable for treatment of the sample. Then each sample was diluted 1:100 at the final concentration of O.D 1 and treated with 10 µL per spot. Overall, topical applications were equally distributed throughout each wounded spot. After applying the sample, mouse was exposed to an infrared heating lamp for 10 minutes so as to be well absorbed in the wounded spots. NFs mixture was applied on mice every day from day1 to 9. 24 hrs later, mice were taken for fluorescence imaging with IVIS® Spectrum CT (PerkinElmer, Singapore Pte Ltd). The field of view, which covered the entire mouse (16 cm), height distance, and optical gain were kept constant throughout the measurement. At the end, the averaged Cy5, Cy3 fluorescence intensity from each region-of-interest was recorded.

The wounds were left to heal naturally for 10 days. On the day 2, 4, 7, and 10, 5 mice in the experimental group were euthanized. After euthanasia, the surrounding skin including the wounded spot was removed and stored at -80° C. for further study.

Monitoring NFs signal on diabetic Mouse model: C57BL/6 mice were anaesthetized using isoflurane and became diabetic by intraperitoneal injection of streptozotocin (STZ, Sigma) at 60 mg/kg in citrate buffer (0.1 M sodium citrate and 0.1 M citric acid, pH 4.5) for 5 consecutive days. The blood glucose levels were measured each day after STZ injection and next 2 continuous weeks by POCT Glucose Detection Kit (GlucoCare). Animals with blood glucose above 250 mg/dL were considered as diabetic ones and were taken for the experimental tests. On the fourth week, the mice were anaesthetized using isoflurane and alfaxalone (30~60 mg/kg) before the wound generation. The wounds were created using the same protocol as that in normal mice. The topical application of SNAs and imaging were conducted similarly as that in the normal mice. All animal experiments were approved by the Institutional Animal Care and Use Committees (IACUC) of Korea University, Korea (KOREA-2021-0107, KOREA-2020-0198).

Statistical Analysis: GraphPad Prism software was used for statistical analysis and graphical representations of data. Statistical tests (t-tests or one-way analysis of variance (ANOVA) were carried out to obtain P value significance. Unless otherwise stated, data are shown as mean ± standard deviation (SD) or percentage of the mean. To assess statistical significance, analysis of variance ANOVA with post-hoc analysis was performed using a web-based statistics calculator from http://astatsa.com/OneWay_Anova_with_TukeyHSD/. No statistical method was used to predetermine sample size. All cell-culture experiments were performed independently at least twice, with multiple duplicate experiments. All in vivo experiments were performed independently with at least 5 subjects. This sample size was sufficient to perform statistical analyses.

### <Example 1> Screening of RNA Biomarkers Related to Various Stages of Wound healing

In an example embodiment of the present disclosure, to identify mRNA biomarkers specific to each cell type, 10 potential mRNA markers for three cell types (i.e., endothelial cells, fibroblasts, and keratinocytes) were identified from the reported 153 candidate pools. The 10 mRNA markers were based on the broad recognition and adaptation of the 10 genes. Then, the cellular expression of the 10 mRNAs was compared to identify a candidate marker showing strong expression in a target cell and weak expression in other types of cells. For cell maintenance, NDF, human keratinocytes (HaCaT), and HUVEC were purchased from Cell Research Corporation (Singapore) and used between 3^{rd} and 10^{th} generation. HUVECs were maintained with Endothelial Cell Growth Medium-2 Bullet Kit (Lonza). All other cells were maintained in DMEM. All media were supplemented with 1% antibiotics (100 U/ml of penicillin and 100 µg/ml of streptomycin) and heat-inactivated 10% fetal bovine serum (FBS) in the presence of 5% CO₂ at 37° C.

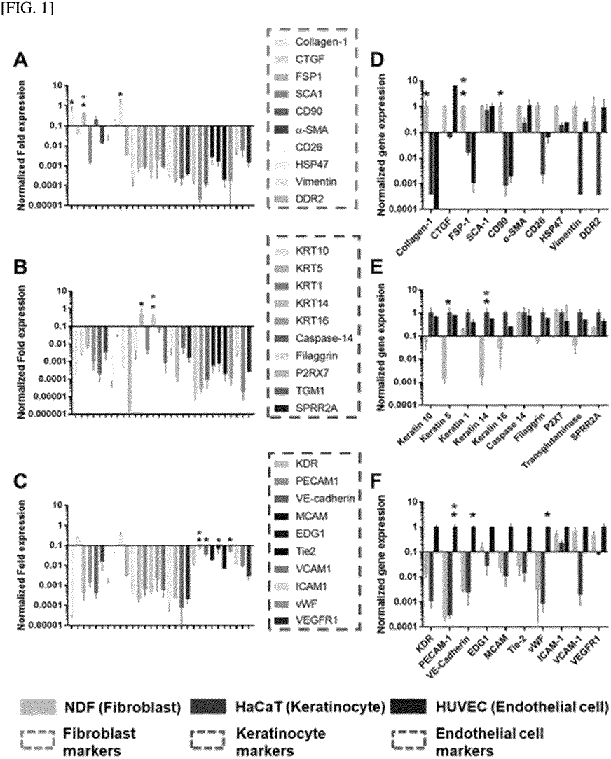

FIGS. 1A-C show the expression of 30 candidate mRNA markers in each cell type, and FIGS. 1D-F show the relative expression of 10 selected mRNA markers in target cells compared to the other two cell types.

As shown in FIGS. 1A and 1D, normal dermal fibroblasts (NDFs) strongly express fibroblast specific protein 1 (FSP-1), collagen-1, and CD90 mRNA among 30 potential biomarkers. FSP-1 has been previously identified as a marker for fibroblasts in wound healing and fibrosis. In addition, the expression of FSP-1 was low in keratinocytes (HaCaT) and endothelial cells (HUVEC). Therefore, in an example embodiment of the present disclosure, FSP-1 was selected as a biomarker for fibroblasts.

Referring to FIG. 1B, keratinocytes (HaCaTs) showed strong expression of mRNA of keratin 5 (KRT5) and keratin 14 (KRT14). KRT14 is specifically expressed in the basal layer of epithelial keratinocytes and has been frequently used as a biomarker in keratinocyte-induced re-epithelialization. Here, KRT14 was selected as a biomarker for keratinocytes in wound healing. It was noted that, as shown in FIG. 1E, KRT14 expression (0.56 fold) was slightly lower in endothelial cells (HUVECs) than in HaCaT.

Referring to FIGS. 1C and 1F, endothelial cells (HUVEC) showed higher expression of PECAM1 (i.e., CD31), KDR, VE-cadherin, and vWF. PECAM1 regulates leukocyte migration and inflammatory vascular responses and is also involved in biological functions including angiogenesis, apoptosis, platelet aggregation, and thrombosis. Therefore, PECAM1, FSP1, and KRT14 were selected as target biomarker genes for endothelial cells (inflammation, angiogenesis, proliferation), fibroblasts (fibrous tissue formation, proliferation), and keratinocytes (proliferation, remodeling), respectively.

### <Example 2> Design, Synthesis, and Characterization of Nanoflare (NF)

qPCR analysis was performed for the selection of target sequences. Cells (NDF, HaCaT, and HUVEC) were seeded in a 6-well plate at a density of 2×10⁴ without stimulation and cultured to reach 80% confluency. Cells were then harvested for total RNA extraction using a PureLink™ RNA mini kit (Thermo Fisher Scientific). 1 µg RNA was reverse transcribed using qScript cDNA SuperMix (Quanta BioSciences). Real-time qPCR was performed on cDNA using Light-Cycler®480 SYBR Green I Master in a CFX96 Touch System (Bio-Rad). As shown in Table 1, primers were purchased from Integrated DNA Technologies. mRNA expression was normalized to GAPDH expression using the 2-ΔΔCt method. Each experiment was performed three times in the same way. To study on growth factor stimulation, cells were first starved for 24 hours in DMEM containing 1% FBS prior to addition of growth factors for 2 days.

TABLE 1

| Protein | Gene | Forward | Sequence List | Reverse | Sequence List |
|---|---|---|---|---|---|
| FSP-1 | S100A4 | 5′-TGAGCAACTTGG ACAGCAACA-3′ | 19 | 5′-ATTTCTTCCTG ggctgcttatct-3′ | 20 |
| CTGF | CCN2 | CAGCATGGACGT TCGTCTG | 21 | AACCACGGTT TGGTCCTTGG | 22 |
| Collagen-1 | COL1A1 | TCTGCGACAACG GCAAGGTG | 23 | GACGCCGGTG GTTTCTTGGT | 24 |
| HSP47 | SERPINH 1 | AACCGTGGCTTC ATGGTGACTC | 25 | TGATGAGGCT GGAGAGCTTG TG | 26 |
| α-SMA | ACTA2 | CTATGCCTCTGG ACGCACAACT | 27 | CAGATCCAGA CGCATGATGG CA | 28 |
| CD24 | CD24 | CACGCAGATTTA TTCCAGTGAAAC | 29 | GACCACGAAG AGACTGGCTG TT | 30 |
| CD26 | DPP4 | AAAGGCACCTG GGAAGTCATCG | 31 | CAGCTCACAA CTGAGGCATG TC | 32 |
| CD90 | THY1 | GAAGGTCCTCTA CTTATCCGCC | 33 | TGATGCCCTC ACACTTGACC AG | 34 |
| SCA-1 | ATXN1 | AGATCGACTCCA GCACCGTAGA | 35 | CTCTACCAAA ACTTCAACGC TGAC | 36 |
| DDR2 | DDR2 | AACGAGAGTGC CACCAATGGCT | 37 | ACTCACTGGC TTCAGAGCGG AA | 38 |
| MMP-2 | MMP2 | AGCGAGTGGAT GCCGCCTTTAA | 39 | CATTCCAGGC ATCTGCGATG AG | 40 |
| Vimentin | VIM | AGGCAAAGCAG GAGTCCACTGA | 41 | ATCTGGCGTT CCAGGGACTC AT | 42 |
| Keratin 10 | KRT10 | 5′-CCTGCTTCAGAT CGACAATGCC-3′ | 43 | 5′-ATCTCCAGGT CAGCCTTGGT CA-3′ | 44 |
| Keratin 14 | KRT14 | TGCCGAGGAATG GTTCTTCACC | 45 | GCAGCTCAAT CTCCAGGTTC TG | 46 |
| Keratin 1 | KRT1 | CAGCATCATTGC TGAGGTCAAGG | 47 | CATGTCTGCC AGCAGTGATC TG | 48 |
| Keratin 5 | KRT5 | GCTGCCTACATG AACAAGGTGG | 49 | ATGGAGAGGA CCACTGAGGT GT | 50 |
| Keratin 16 | KRT16 | CTACCTGAGGAA GAACCACGAG | 51 | CTCGTACTGG TCACGCATCT CA | 52 |
| Involucrin | IVL | GGTCCAAGACAT TCAACCAGCC | 53 | TCTGGACACT GCGGGTGGTT AT | 54 |
| Filaggrin | FLG | GCTGAAGGAACT TCTGGAAAAGG | 55 | GTTGTGGTCT ATATCCAAGT GATC | 56 |
| Transglutamin ase | TGM1 | GAACGACTGCTG GATGAAGAGG | 57 | CTTGATGGAC TCCACAGAGC AG | 58 |
| P2X purinoceptor 7 | P2RX7 | CGACTAGGAGA CATCTTCCGAG | 59 | GCAGTGATGG AACCAACGGT CT | 60 |
| P2Y purinoceptor 2 | P2RY2 | CGAGGACTTCAA GTACGTGCTG | 61 | GTGGACGCAT TCCAGGTCTT GA | 62 |
| Caspase 14 | CASP14 | GGTGGATGTGTT CACGAAGAGG | 63 | CCTTCTTGAA CCAGCTCTGC TTC | 64 |
| SPRR2A | SPRR2A | CAGTGCCAGCAG AAATATCCT | 65 | CCAAATATCC TTATCCTTTCT TGG | 66 |
| PECAM-1/CD31 | PECAM1 | 5′-AAGTGGAGTCCA GCCGCATATC-3′ | 67 | 5′-ATGGAGCAGG ACAGGTTCAG TC-3′ | 68 |
| KDR/VEGFR 2 | KDR | GGAACCTCACTA TCCGCAGAGT | 69 | CCAAGTTCGT CTTTTCCTGG GC | 70 |
| VEGFR1 | FLT1 | CCTGCAAGATTC AGGCACCTATG | 71 | TTGCAGTGCT CACCTCTGAT T | 72 |
| CD34 | CD34 | CCTCAGTGTCTA CTGCTGGTCT | 73 | GGAATAGCTC TGGTGGCTTG CA | 74 |
| Endoglin/CD1 05 | ENG | CGGTGGTCAATA TCCTGTCGAG | 75 | AGGAAGTGTG GGCTGAGGTA GA | 76 |
| MCAM/CD14 6 | MCAM | ATCGCTGCTGAG TGAACCACAG | 77 | CTACTCTCTG CCTCACAGGT CA | 78 |
| ICAM-1 | ICAM1 | AGCGGCTGACGT GTGCAGTAAT | 79 | TCTGAGACCT CTGGCTTCGT CA | 80 |
| VCAM-1 | VCAM1 | GATTCTGTGCCC ACAGTAAGGC | 81 | TGGTCACAGA GCCACCTTCT TG | 82 |
| VE-Cadherin/CD1 44 | CDH5 | GAAGCCTCTGAT TGGCACAGTG | 83 | TTTTGTGACTC GGAAGAACTG GC | 84 |
| vWF | VWF | CCTTGAATCCCA GTGACCCTGA | 85 | GGTTCCGAGA TGTCCTCCAC AT | 86 |
| Tie-2 | TEK | GGTCAAGCAACC CAGCCTTTTC | 87 | CAGGTCATTC CAGCAGAGCC AA | 88 |
| EDG1/S1PR1 | S1PR1 | CCTGTGACATCC TCTTCAGAGC | 89 | CACTTGCAGC AGGACATGAT CC | 90 |

In an example embodiment of the present disclosure, after identification of target genes corresponding to each stage of wound healing, nanoflares targeting these mRNA biomarkers were synthesized as follows. The nanoflare consisted of a 13 nm gold nanoparticle (GNP) core and surrounding nucleic acid duplexes. The duplex includes a recognition sequence (named R) and a flare sequence (named F). The R/F ratio was optimized by checking hybridization efficiency thereof. As shown in FIG. 2, the higher the R:F ratio, the higher the hybridization might be. Although the 10:1 R/F ratio showed the highest R/F hybridization, in an example embodiment of the present disclosure, a 5:1 R/F ratio was selected for subsequent synthesis in order to balance hybridization efficiency and assembly cost.

Table 2 below lists a thiol-modified recognition sequence (named R) and a flare sequence (named F) whose 5′ end is modified with a fluorescent dye, and the recognition sequences are complementary to the target sequence. In order to optimize the sensitivity and stability of the nanoflare, the predicted free energy change of all R-F duplexes was set to ≥20 kcal/mol, and R-T (recognition-target) to ≤60 kcal/mol as shown in Table 3 below.

TABLE 2

| Gene | Nucleotide | | Sequence | Sequence list |
|---|---|---|---|---|
| FSP1 | Human Recognition | | 5′-ATTTCTTCCTGGGCTGCTTATCTGGGAAAA-AAAA-SH 3′ | 1 |
| | Mouse Recognition | | 5′-ACTTCTTCCGGGGCTCCTTATCTGGGCAAA-AAAA-SH 3 | 2 |
| | Flare | | | |
| | Length | Human 18 nt | 3′- CCCGACGAATAGACCCTT- FAM -5′ | 3 |
| | | Human 16 nt | 3′-CGACGAATAGACCCTT- FAM-5′ | 4 |
| | | Human 15 nt | 3′- GACGAATAGACCCTT- FAM -5′ | 5 |
| | | Mouse 15 nt | 3′- GAGGAATAGACCCGT- Cy3 -5′ | 6 |
| | | Human 14 nt | 3′- ACGAATAGACCCTT- FAM -5′ | 7 |
| | | Human 12nt | 3′- GAATAGACCCTT- FAM -5′ | 8 |
| PECAM 1 | Human Recognition | | 5′-ATGGAGCAGGACAGGTTCAGTCTTTCAC AAAAAA-SH-3′ | 9 |
| | Mouse Recognition | | 5′-TTAAGGGAGCCTTCCGTTCTTAGGGTCGA CAAAAAA-SH-3′ | 10 |
| | Human Flare | | 3′-CCAAGTCAGAAAGTG-Cy3-5′ | 11 |
| | Mouse Flare | | 3′-CAAGAATCCCAGCTG-Cy3-5′ | 12 |
| KRT14 | Human Recognition Same as human | | 5′-GACTGCAGCTCAATCTCCAGGTTCTGCAA AAAAA-SH -3′ | 13 |
| | Human Flare Same as human | | 3′- AGAGGTCCAAGACGT- Cy3 -5′ | 14 |
| GAPDH | Human Recognition | | 5′-GATGGCATGGACTGTGGTCATGAGTCCTA AAAAA-3′ | 15 |
| | Mouse Recognition | | 5′-GATGGCATGGACTGTGGTCATGAGCCCTA AAAAA-3′ | 16 |
| | Human Flare | | 3′-CACCAGTACTCAGGA-Cy5-5′ | 17 |
| | Mouse Flare | | 3′-CACCAGTACTCGGGA-Cy5-5′ | 18 |

TABLE 3

| Flare | -ΔG(Duplex) (kcal/mol) | |
|---|---|---|
| | FSP1 (human) | FSP1 (mouse) |
| 12 nt | 13.7 | |
| 14 nt | 18.9 | |
| 15 nt | 18.7 | 21.4 |
| 16 nt | 21.0 | |
| 18 nt | 24.5 | |
| Target | 53.4 | 58.6 |
| | PECAM1 (human) | PECAM1 (mouse) |
| 15 nt | 19.4 | 21.4 |
| Target | 48.9 | 61.4 |
| | KRT14 (human) | KRT14 (mouse) |
| 15 nt | 20.6 | 20.8 |
| Target | 56.8 | 56.8 |
| | GAPDH (human) | GAPDH (mouse) |
| 15 nt | 19.6 | 20.9 |
| Target | 57.6 | 59.9 |

*Predicted using RNA Structure

As shown in FIG. 2, the recognition strand (20 µL, 100 µM) and the flare strand (10 µL, 100 µM) were subjected to annealing in different volume ratios (1:1, 2:1, 5:1, and 10:1, v/v) from 95° C. to room temperature (RT) for at least 1 hour. Before purification of the produced R-F duplex with Micro Bio-Spin 6 Columns (Bio-Rad), 50 mM of TCEP was treated at room temperature for 1 hour to activate the thiol-modified recognition strand. A mixture containing purified R-T duplexes was added to 13 nm citrate-capped GNPs (1 mL, 5 nM) in a volume ratio of 100:700. The mixture was then stored at -20° C. for 2 hours, and 50 µL of 1 M NaCl was added every 30 min until a final concentration of NaCl reached 0.3 M. After each addition of NaCl, the solution was sonicated for 30 seconds. After stirring overnight, functionalized GNPs or nanoflares were induced after centrifugation at 14,000 g for 30 min. Nanoflares were washed twice with 0.05% Tween-20 in PBS. Concentrations thereof were determined by absorbance at 520 nm (Nanodrop 2000, Thermo Fisher Scientific). The hydrodynamic size and zeta potential of spherical nucleic acid (SNA) were measured using a dynamic light scattering system (Zetasizer, Malvern, UK).

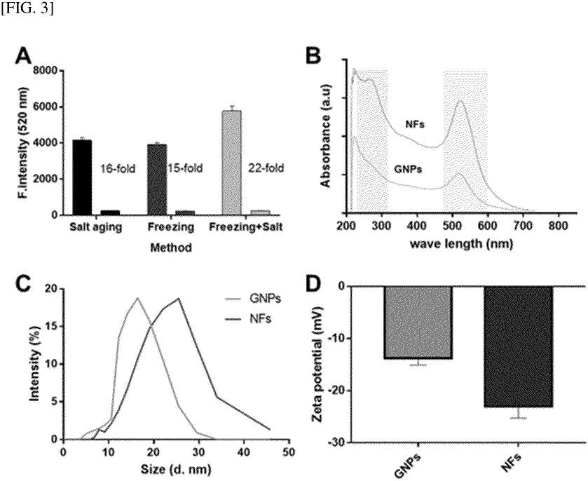

Nanoflares were synthesized via a modified salt aging method in which some features of low-temperature assembly methods are integrated. The method provided the most sensitive nanoflare which showed the greatest change in the fluorescence signal upon addition of the target sequence (FIG. 3A). The sharp absorption by the nanoflare at 260 nm came from the nucleic acid duplex of the nanoflare (FIG. 3B). Assembly of R-F duplexes on the GNP core also increased the hydrodynamic size from 13 nm to 28 nm (FIG. 3C) and decreased the zeta potential from -14 mV to -24 mV (FIG. 3D).

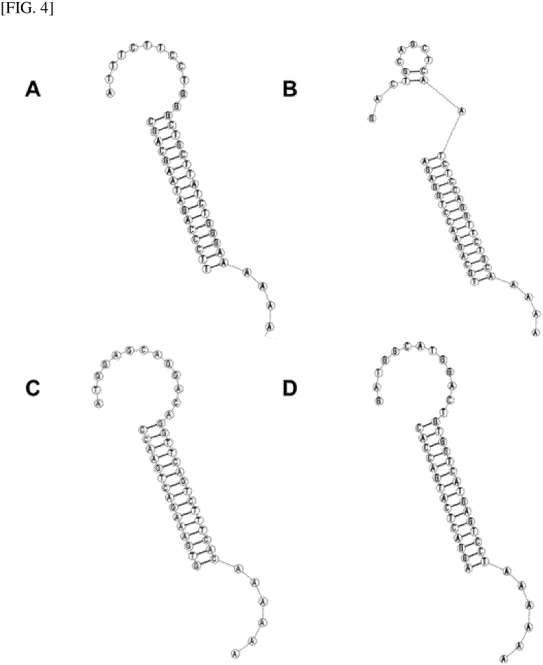

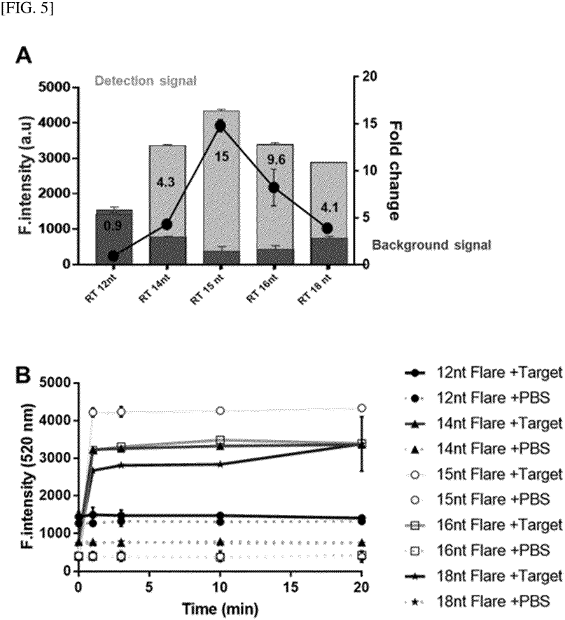

In order to optimize the sensitivity and stability of the nanoflare, the effect of the length of the flare sequence was studied by investigating the change in fluorescence intensity and Gibbs free energy during R-F hybridization as shown in Table 3 and FIG. 4. Using the FSP1 nanoflare as a model, in an example embodiment of the present disclosure, the complementary flare sequence was changed from 122 nucleotides to 18 nucleotides. Corresponding changes in Gibbs free energy due to hybridization were calculated by RNA structure bifold software. As shown in Table 3, the longer flares induced greater changes in Gibbs free energy, indicating higher stability of the R-F duplex. In an example embodiment of the present disclosure, fluorescence restoration was further investigated when the target sequence was added to an FSP1-NF solution having different flare sequence length, which is shown in FIG. 5. As shown in FIG. 5A, all FSP-1 nanoflares except for 12 nucleotides successfully restored the fluorescence signal. Referring to FIG. 5B, the fluorescence restoration was efficient and occurred rapidly within 2 minutes. Since the FSP-1 nanoflares having 15 nucleotide-long flares showed the biggest changes in the fluorescence signal as well as the lowest background signal (compared to 0.1, 4.3, 15, 9.6, and 4.1 fold for nucleotide flares 12, 14, 15, 16, and 18, respectively), the 15 nucleotide-long flare was selected for the subsequent synthesis of nanoflares in an example embodiment of the present disclosure.

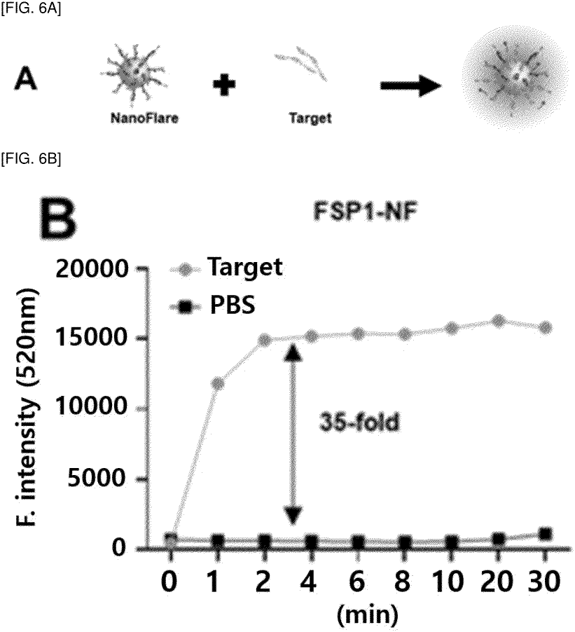

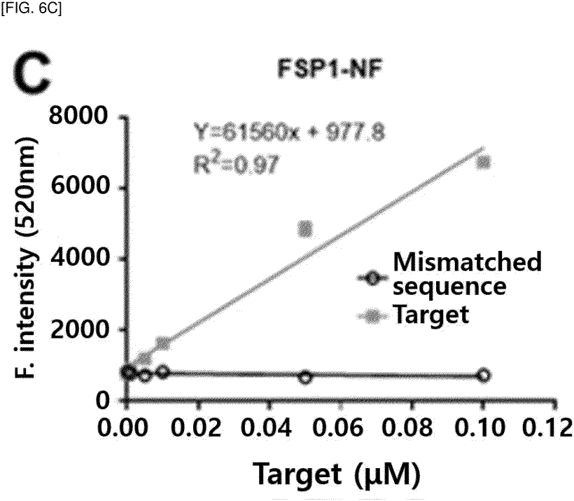

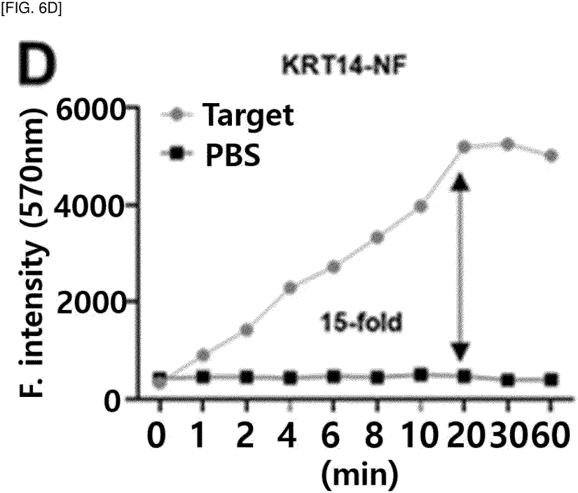

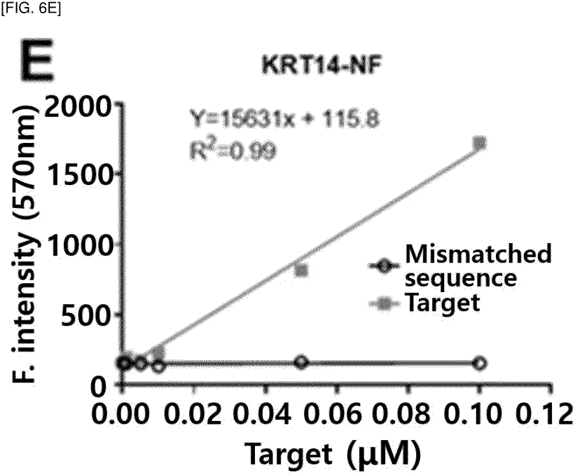

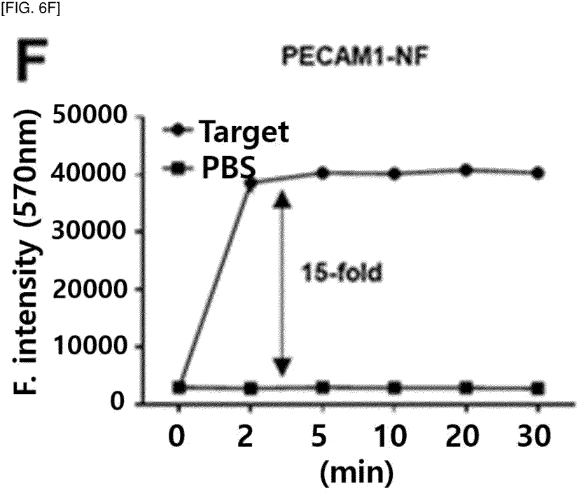

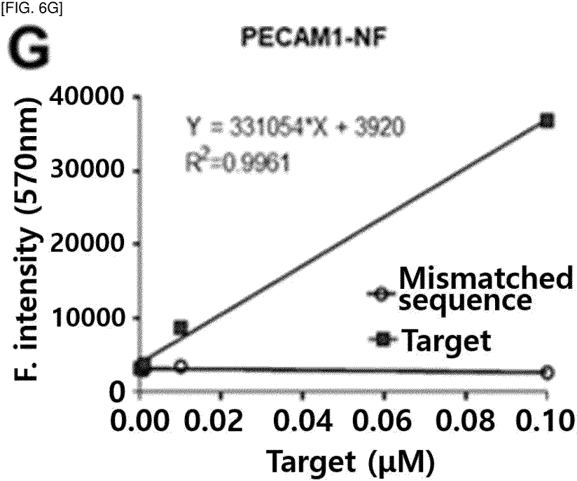

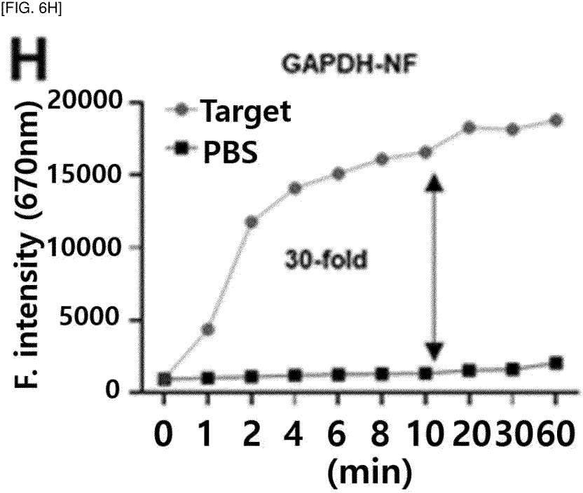

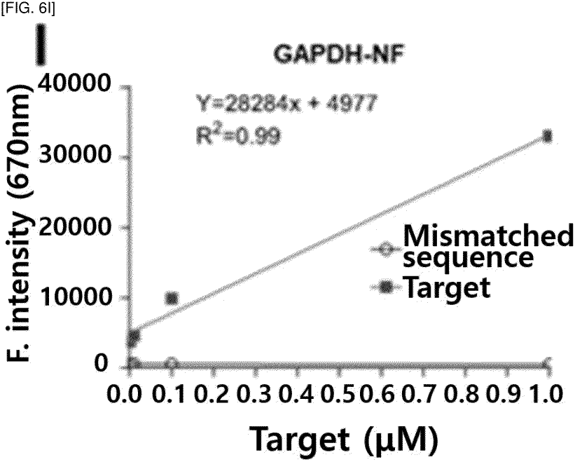

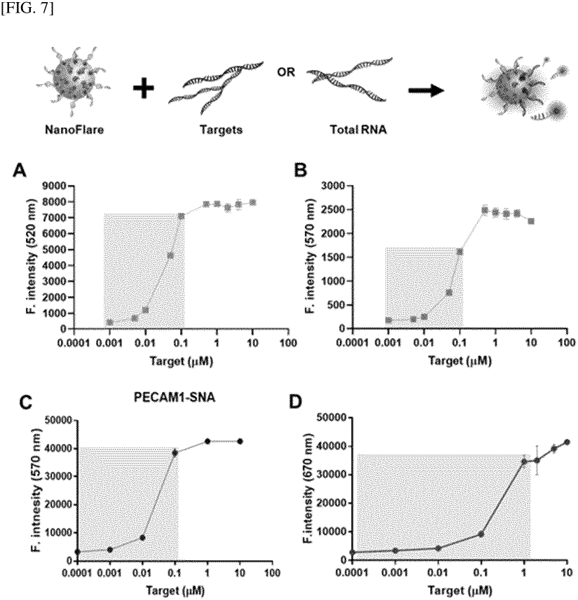

As mentioned above, PECAM1, FSP1, and KRT14 were selected as target biomarkers for endothelial cells (inflammation, angiogenesis, proliferation), fibroblasts (fibrous tissue formation, proliferation), and keratinocytes (proliferation, remodeling). Therefore, in an example embodiment of the present disclosure, nanoflares targeting the genes were synthesized using an optimized synthesis protocol. Nanoflares recognizing the GAPDH reference gene were also synthesized with the same protocol to function as control signals as well as cellular quantification. Referring to FIGS. 6A to 6I, all the nanoflares showed excellent sensitivity to each target sequence. For example, referring to FIG. 6A, there was a change in fluorescence intensity at least 35 fold upon binding between FSP1-NF and the target sequence. The fluorescence signal showed a linear correlation with the concentration of the target sequence in the range of 0.001-0.1 µM (R2 = 0.97) (FIGS. 6B and 7A). Referring to FIG. 7A, signal saturation was observed only when the target concentration was GNP O.D of 0.4 to 0.1 µM or more. Similar results were obtained for KRT14-NF (FIGS. 6C and D, FIG. 7B, R2 = 0.99). On the other hand, PECAM1-NF (FIGS. 6E and F, FIG. 7C) and GAPDH-NF (FIGS. 6G and H, FIG. 7D) showed better sensitivity having good correlations (R2 = 0.996 and 0.98, respectively) and target reactivity between 0.0001 and 0.1 µM. All nanoflares showed satisfactory selectivity, monitoring window, and specificity. As shown in FIGS. 6B, D, F, and H, the nanoflare responded only to the target sequence although the signal recovery rate was slightly different.

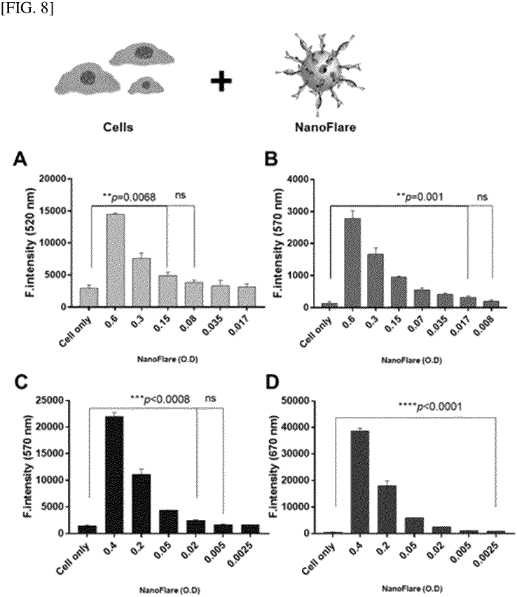

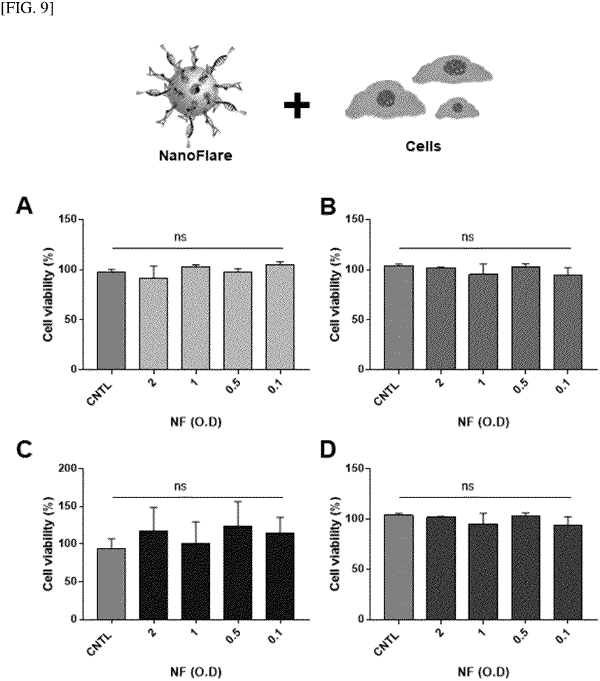

As shown in FIG. 8, the nanoflare working concentration for intracellular experiments was further optimized to facilitate subsequent use in in-vitro and in-vivo models. Reliable fluorescence changes were detected (0.08-0.6 for FSP1-NF, O.D. 0.01-0.6 for KRT14-NF, O.D. 0.01-0.4 for PECAM1-NF, and 0.005-0.4 for GAPDH-NFs). Then, in an example embodiment of the present disclosure, the nanoflare concentration was normalized for subsequent experiments, that is, O.D 0.1 for FSP1-NF and 0.0125 for KRT14-NF, PECAM1-NF, and GAPDH-NF (concentration of FSP1-NF was higher than others due to intensity of a dye). Additionally, as shown in FIG. 9, cytotoxicity analysis revealed that all nanoflares were non-toxic up to a concentration of O.D 0.4.

### <Example 3> Monitoring of Dynamic Changes of Cellular mRNA Target With Nanoflare without GAPDH normalization

For hybridization of the nanoflare and target RNA in cellular RNA extracts, RNA extraction from 2D cultured cells was performed using a PureLink™ RNA mini kit (Thermo Fisher Scientific). The nanoflare was then mixed with total cellular RNA extract for 30 min at room temperature. The fluorescence intensity of the mixture was investigated using Synergy HT Microplate Reader (BioTek). Each measurement was repeated three times in the same way.

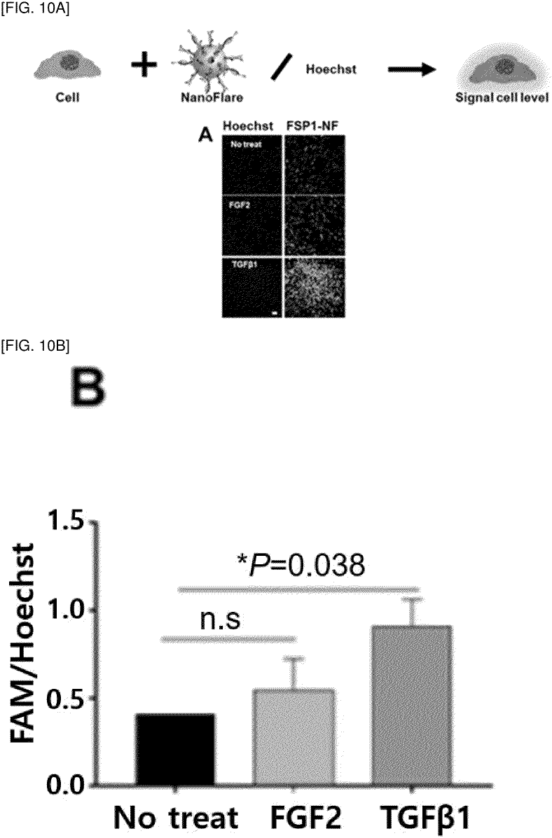

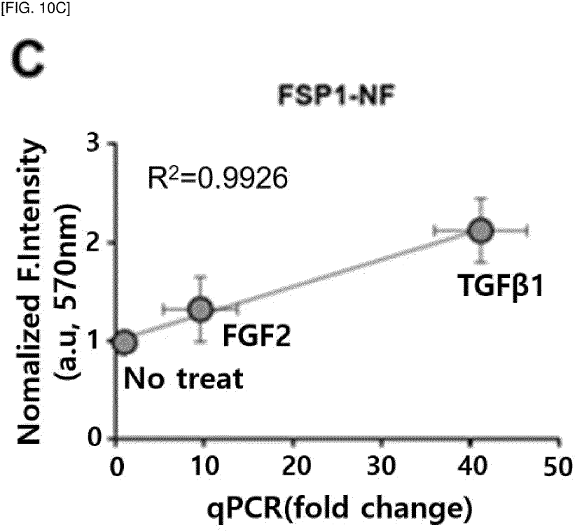

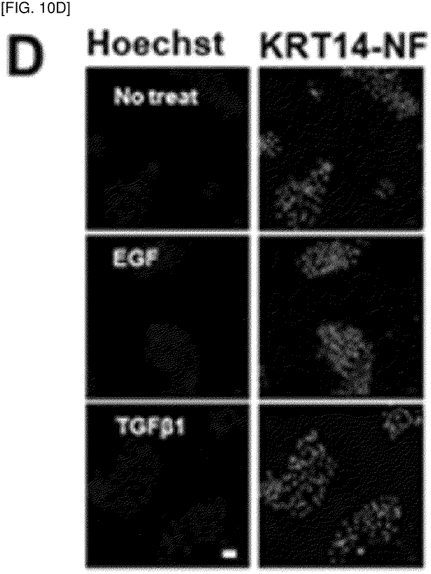

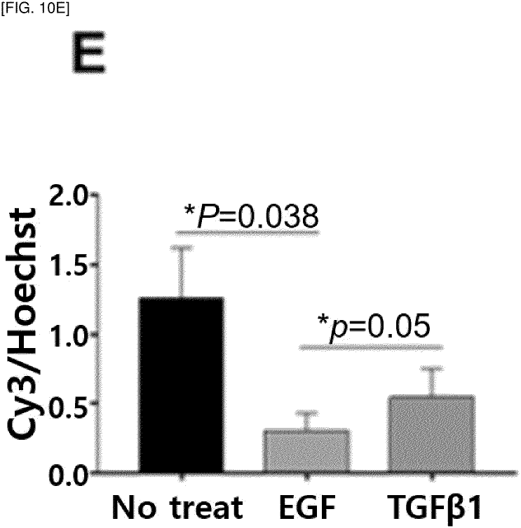

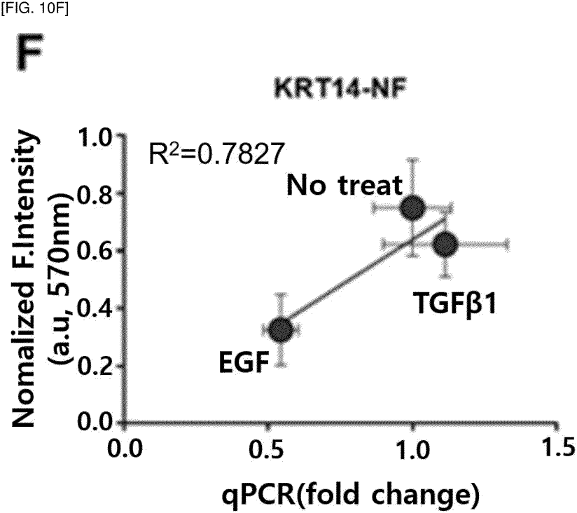

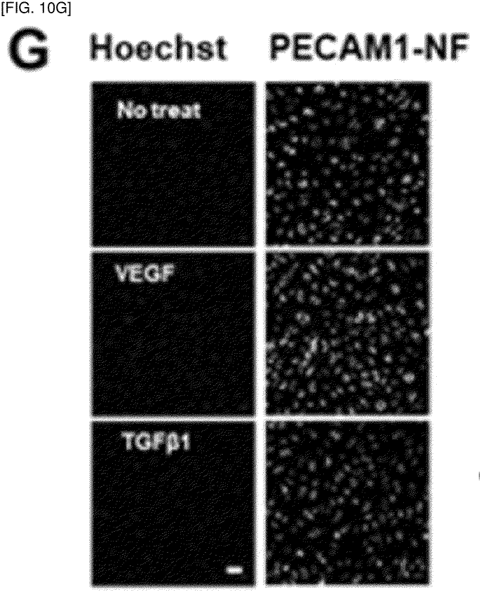

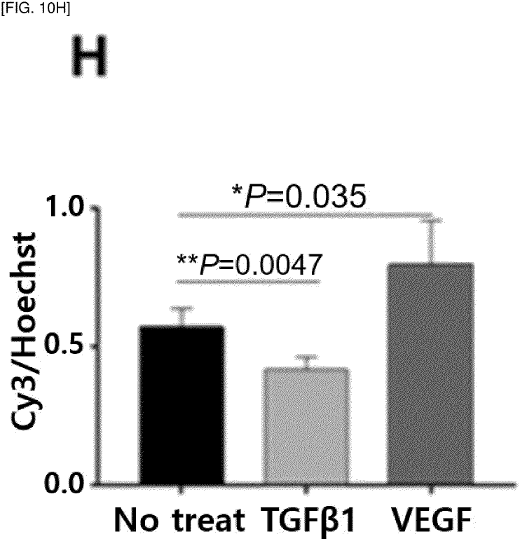

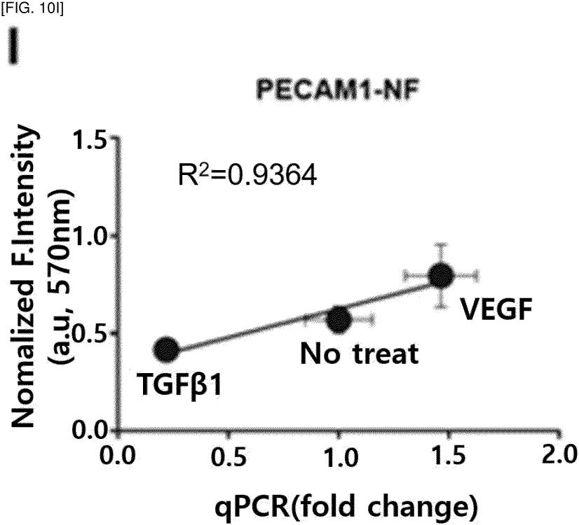

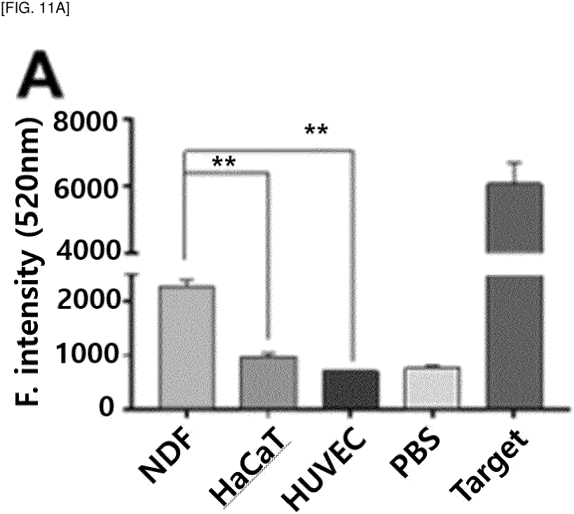

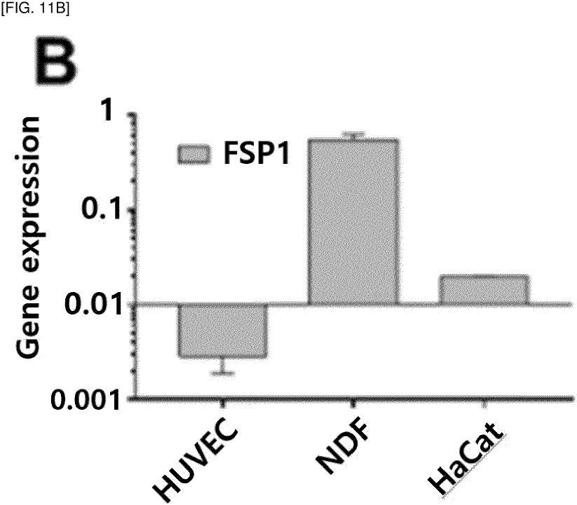

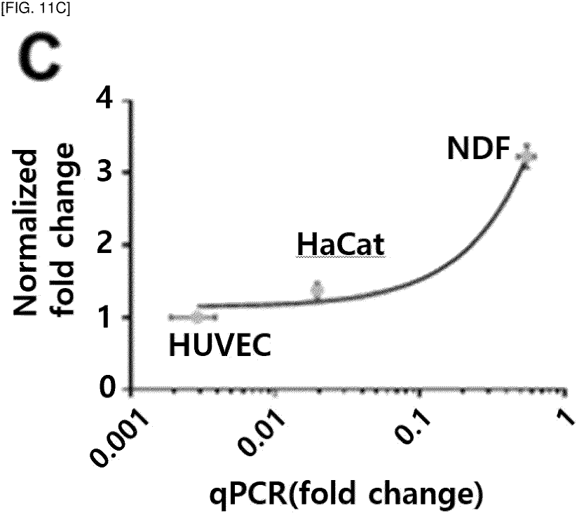

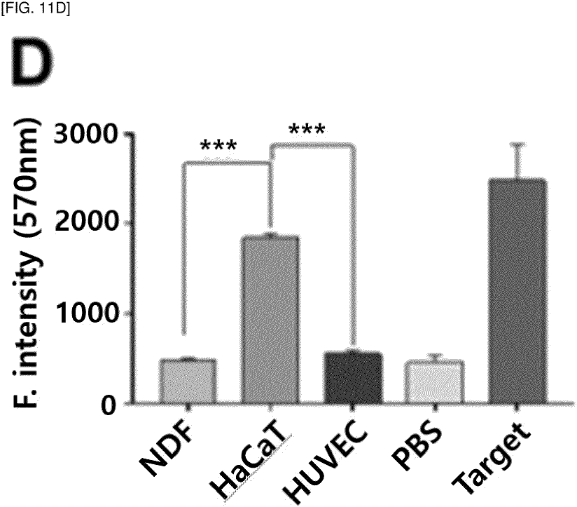

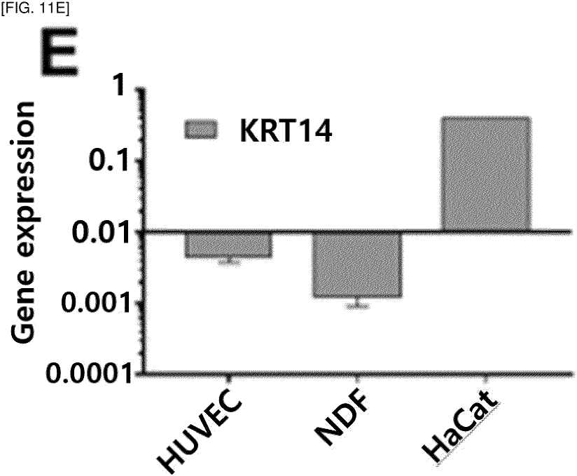

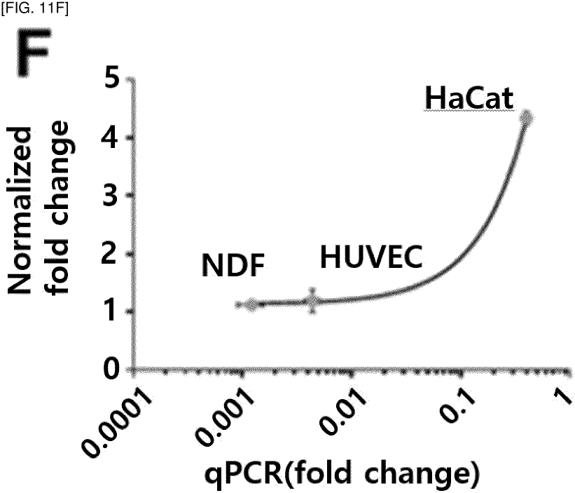

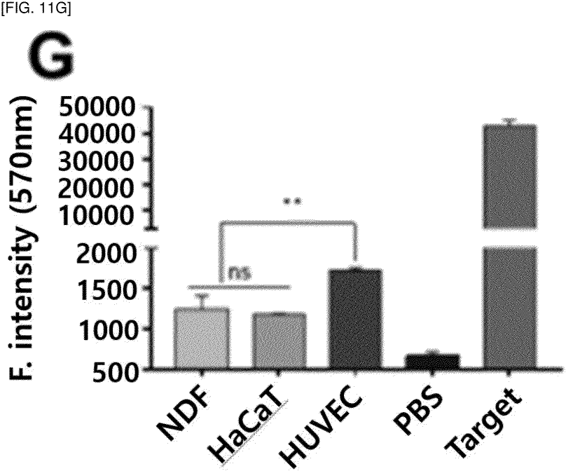

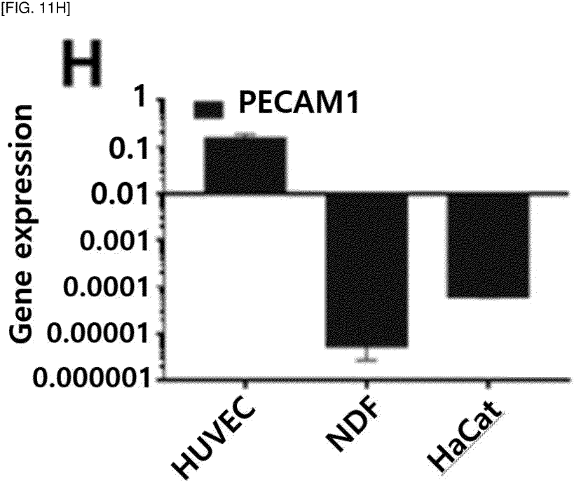

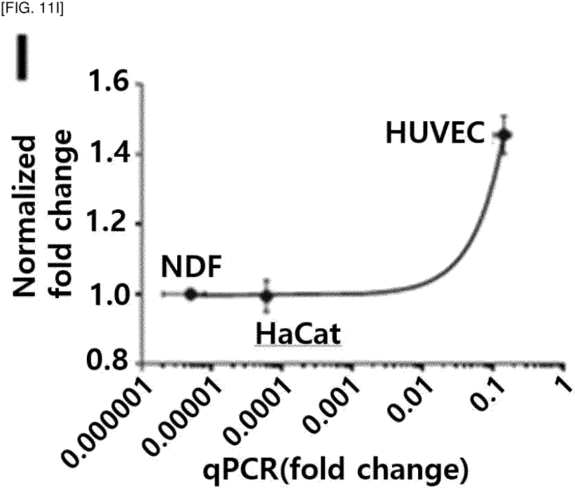

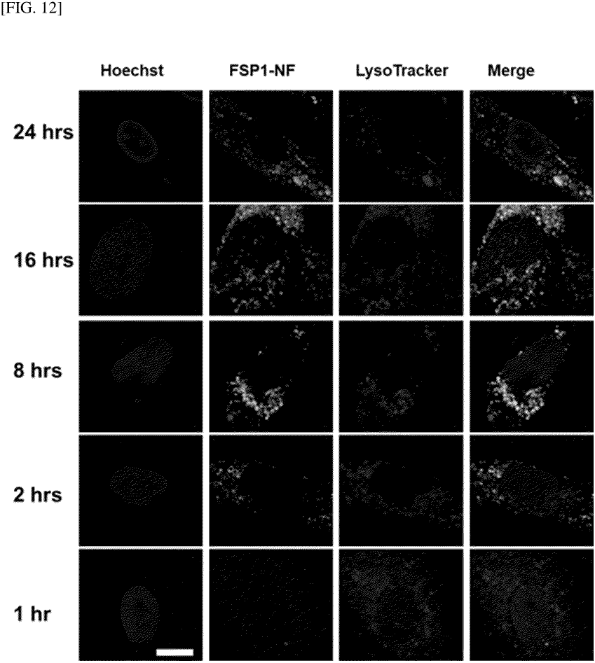

To evaluate the dynamic performance of the nanoflare, NDF (fibroblasts) was stimulated with TGFβ1 and FGF2, HaCaT (keratinocytes) with TGFβ1+EGF, and HUVECs (endothelial cells) with TGFβ1+VEGF. Cells were then labeled with a relevant nanoflare to be imaged. Fluorescence intensity was quantified at the single cell level via normalization by Hoechst (i.e., nanoflare signal/Hoechst). In the case of NDF (FIG. 10A), treatment of both TGFβ1 and FGF2 increased the normalized fluorescence signal of FSP1-NF (2.2±0.15 fold and 1.33±0.17 fold, respectively) (FIG. 10B). As shown in FIG. 10C, qPCR analysis showed correlated upregulation of FSP-1 mRNA expression (41±5 fold and 9±5 fold by treatment of TGFβ1 and FGF2, respectively). Referring to FIG. 11A, the expression of FSP-1 in various types of cells was further investigated using FSP1-NF. NDF showed the highest signal (3.1±0.15 fold) compared to HUVEC which showed the lowest expression. The results were well matched up with the qPCR results as shown in FIGS. 11B and C. SNA-based nanoflares are generally recognized by class A scavenger receptors and internalized via the endosomal pathway. According to the existing method, SNA is primarily localized in early endosomes after 1-2 hours of incubation with cells. Here, in an example embodiment of the present disclosure, it was found that the nanoflare was absorbed by cells through endocytosis after incubation for 2 hours. As shown in FIG. 12, the signal from the nanoflare overlapped with lysosome.

In an example embodiment of the present disclosure, for the evaluation of KRT14-NF, HaCaT cells were treated with TGFβ1 and EGF. As shown in FIG. 10D, TGFβ1 treatment did not derive a significant change in KRT14 expression, whereas EGF induced a 50% decrease in KRT14 as shown in FIG. 10E. This was determined by qPCR analysis as shown in FIG. 10F. As shown in FIGS. 11D to 11F, the expression of KRT-14 in other types of cells was further examined using KRT14-NF, and as expected, HaCaT showed the highest expression (4.3±0.11 fold).

PECAM1-NF was finally used to track the effect of TGFβ1 and VEGF treatment in HUVECs (FIGS. 10G-H). The fluorescence signal from cellular PECAM1-NF was decreased to 20±3% in the TGFβ1-treated group, but increased by 30±5% in the VEGF-treated group. Consistent with FSP1-NF and KRT14-NF, qPCR analysis proved the observation as shown in FIG. 10I. As shown in FIG. 11G, PECAM1-NF was also used to detect the expression level of PECAM1 in NDF, HaCaT, and HUVEC. These experiments showed that PECAM1-NF showed the highest signal in HUVECs (1.45±0.01 fold) corresponding to a gene expression pattern detected by qPCR (FIGS. 11H to 11I).

Taken together, the nanoflare enabled the monitoring of dynamic cellular mRNA without signal normalization to a reference gene.

### <Example 4> Growth Factor Screening Using the Nanoflare to Monitor Target Genes by GAPDH normalization

To quantify the cellular expression of target genes, cells in 48-well plates were subjected to starvation for 24 hours in DMEM containing 1% FBS. Later, FGF2/TGFβ1, EGF/TGFβ1, and VEGF/TGFβ1 were added to the media for NDF, HaCaT, and HUVEC, respectively. After 2 days, the cells were washed with PBS and fed with 5% FBS medium containing the nanoflare (concentrations were O.D 0.125 and 0.1). After 16 hours, cells were stained with Hoechst 33342 and subjected to confocal imaging (LSCM, Carl Zeiss). In addition, after extraction of total RNA from the same experimental group, the extract was treated with the nanoflare prior to fluorescence intensity measurement. qPCR was also performed with the extracted RNA for comparison with results of the nanoflare quantification. Comparison of target gene expression in NDF, HaCaT, and HUVEC was conducted without stimulation in a similar manner.

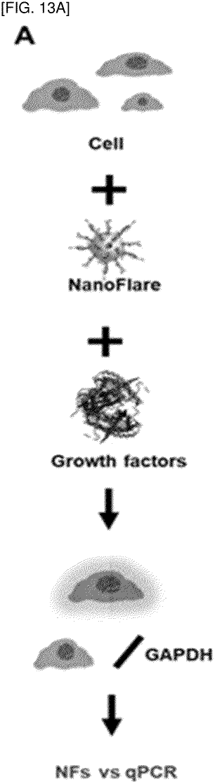

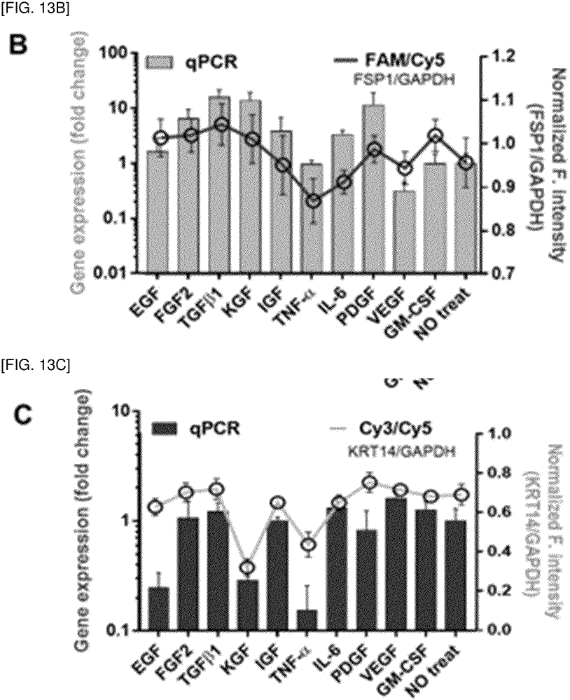

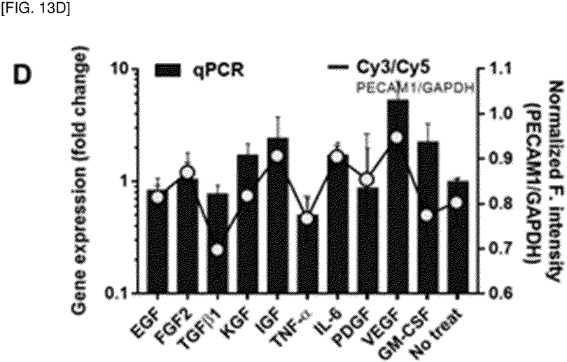

As shown in FIG. 13A, the ability of the nanoflare was further investigated to analyze the stimulatory effect of growth factors having unclear effects on the three types of cells. In an example embodiment of the present disclosure, 10 growth factors known to be involved in wound healing were selected. Growth factors include EGF, FGF2, IGF, KGF, PDGF, TGF β1, VEGF, GM-CSF, IL-6, and TNF-α. Here, GAPDH mRNA was adopted as a reference gene and monitored for qPCR comparison.

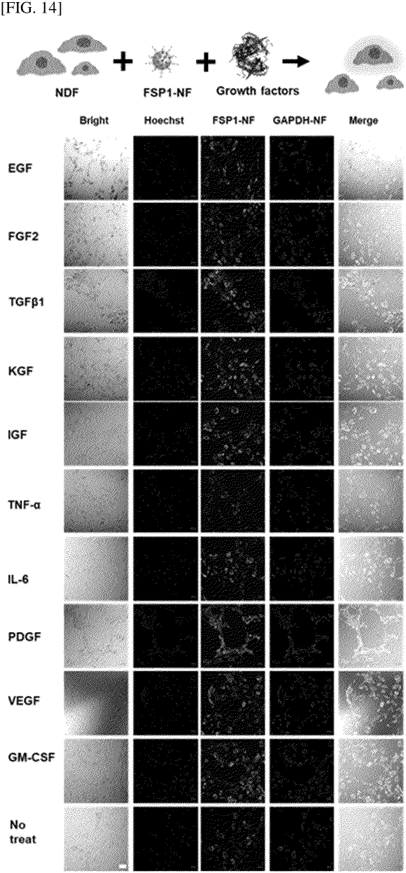

FSP-1 expression in fibroblasts (NDF) was increased by TGFβ1 (8%), FGF2 (7%), KGF (7%), PDGF (3%), GM-CSF (7%), and EGF (5%). On the other hand, IGF and VEGF did not induce any significant change. Conversely, referring to FIG. 13B (red line) and FIG. 14, TNF-α and IL-6 decreased FSP-1 expression by 10% and 4%, respectively. Referring to FIG. 13B (green bar), qPCR analysis confirmed results that FSP1 expression was the highest after treatment of TGFβ1 and the lowest after treatment of VEGF and TNF-α.

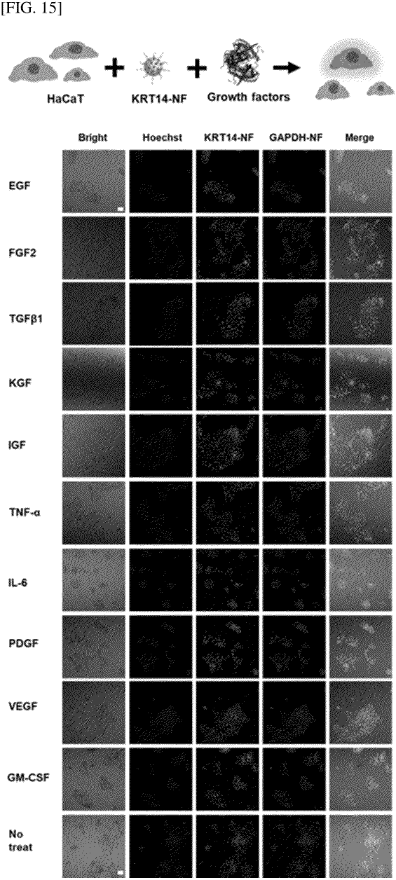

qPCR analysis in keratinocytes (HaCaT) showed no change in KRT14 expression after treatment of FGF2, TGFβ1, IGF, and GM-CSF. However, EGF (0.24 fold), KGF (0.28 fold), and TNF-α (0.15 fold) down-regulated KRT14 expression, whereas IL-6 slightly upregulated KRT14 expression (FIG. 13C, red bar). In an example embodiment of the present disclosure, there was no change after treatment of FGF2, TGFβ1, IGF, VEGF, and GM-CSF, and the reduced nanoflare signal was observed in samples treated with EGF (8%), KGF (54%), and TNF-α (37%) (FIG. 15, FIG. 13C, green line). Therefore, the results of qPCR and nanoflare were quite consistent.

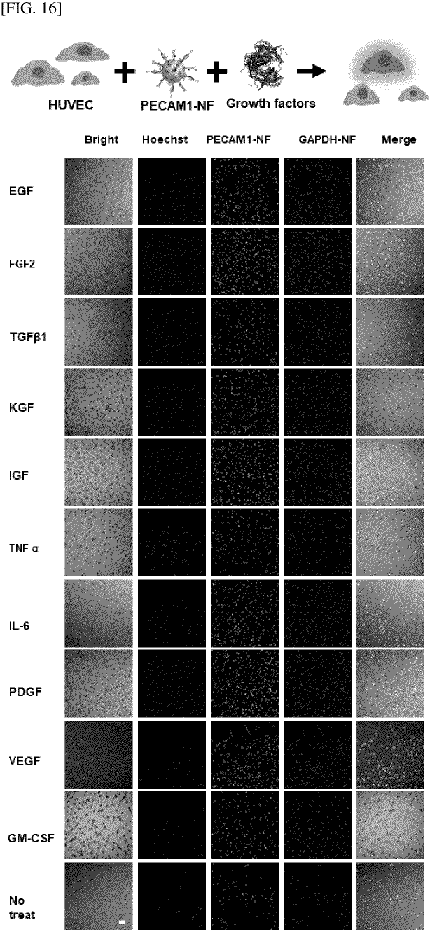

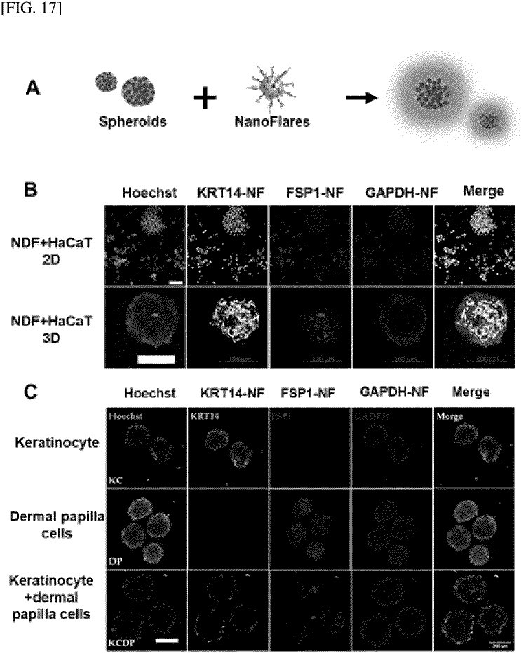

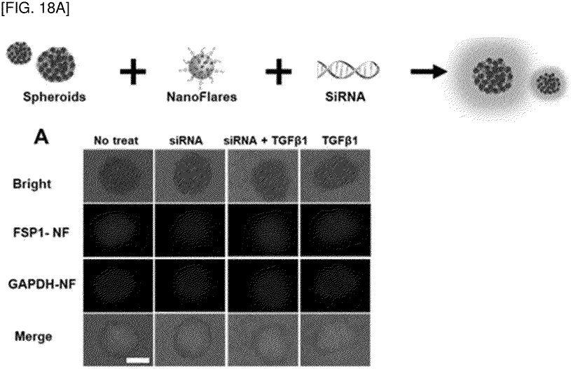

After treatment of FGF2, IGF, IL-6, and VEGF in endothelial cells (HUVEC), the fluorescence signal of PECAM1-NF increased, whereas TGFβ1, TNF-α, and GM-CSF down-regulated PECAM1 signal (FIG. 16, FIG. 13D, black line). qPCR confirmed that TGFβ1 and TNF-α decreased while KGF, IGF, IL-6, and VEGF increased PECAM1 expression (FIG. 13D, blue bar).

Taken together, these results confirmed the efficacy of the nanoflare in monitoring the dynamic changes of target genes in various types of skin-related cells.

### <Example 5> Nanoflare Penetration and Specific Labeling of Cells in 3D Cell Spheroids

To identify potent growth factors, cells were cultured in 48-well plates and starved for 24 hours before addition of individual growth factors, followed by incubation for 48 hours (EGF: 40 ng/mL, FGF2: 40 ng/mL, TGFβ1: 40 ng/mL, KGF: 40 ng/mL, IGF: 50 ng/mL, TNFα: 40 ng/mL, IL-6: 40 ng/mL, PDGF: 8 ng/mL, VEGF: 20 ng/mL, GM-CSF: 20 ng/mL, in 1% FBS/DMEM). Next, DMEM containing O.D 0.1 FSP1-NF, O.D 0.0125 KRT14-NF, PECAM1-NF, and GAPDH-NF was further added for 16 hours. After washing, 1 mL of Hoechst 33342 working solution was added for 30 minutes to stain the cell nucleus. Cells were washed twice with PBS and imaged with a confocal microscope (Carl Zeiss). Laser intensity and exposure were kept constant at the interval of experiments performed at room temperature with minimal background light.

3D spheroid cultures were prepared using immortalized normal human keratinocytes (KC, N/TERT-1, permitted by Professor JG Rheinwald) and human primary follicular dermal papilla cells (DPC, Adult, Cell Application Inc.). KC and DPC were grown in keratinocyte serum-free medium (KSFM) and DMEM supplemented with 10% fetal bovine serum (Hyclone) and 0.14 mg/mL bone pituitary extract (Gibco), respectively. Single cell suspensions were seeded in low adhesion 96-well plates (Corning) and incubated at 37° C. for spheroid formation.

To prepare spheroids, 2000 KC, 2000 DPC, and 2000 KC + 2000 DPC were inoculated in each well to prepare KC, DP, and KCDP spheroids. Spheroids were collected 3 days post-seeding and incubated, for 16 hours, in 100 µL of 1% FBS DMEM containing a mixture of KRT14-NF, FSP1-NF, and GAPDH-NF (2-5 µL/mL at concentrations of OD 0.1-0.125). The spheroids were then washed once with PBS and stained with Hoechst 33342 to stain the nuclei. Signals of KRT14-NF, FSP1-NF, and GAPDH-NF were visualized on an Olympus FV3000RS inverted confocal microscope using Cy3, 6-FAM, and Cy5 filters, respectively. Z-stacks were collected for all samples and processed with FIJI software to obtain sum slice projection images.

For small interfering RNA (siRNA) transfection for 3D spheroids, 2000 NDF, 2000 HaCaT, and 2000 NDF + 000 HaCaT per well were inoculated on day 0 to prepare spheroids for use in nanoflare sensitivity analysis. On day 1 after inoculation, spheroids were transfected with 10 µM of TGFBR1 siRNA duplex (Thermofisher Scientific) using Lipofectamine® 2000 (ThermoFisher Scientific) according to the manufacturer’s protocol and incubated at 37° C. for 24 hours. Then, the transfected medium was replaced with 100 µL of 1% FBS DMEM with or without TGFβ1 (10 ng/mL) and incubated at 37° C. for 24 hours. After the treatment, spheroids were incubated in 100 µL of 1% FBS DMEM containing either KRT14-NF and GAPDH-NF (HaCaT spheres) or FSP1-NF and GAPDH-NF (NDF spheres) for another 16 hours. Then, the spheroids were washed once with PBS and stained with Hoechst 33342. A Cy3 filter was used to visualize the signals of KRT14-NF and FSP1-NF, where the GAPDH-NF signal was detected using a Cy5 filter on an Olympus FV3000RS inverted confocal microscope. Z-stacks were collected for all samples and processed using FIJI software to obtain sum slice projection images.

As shown in FIG. 17A, the nanoflares were further examined in 2D and 3D co-culture models. In an example embodiment of the present disclosure, a different strategy was used to co-culture NDF and HaCaT and to prepare 2D and 3D models. As shown in FIG. 17B, the nanoflares introduced in both models were able to successfully identify target genes in specific cells. For example, FSP1-NF fluorescence was only visible in NDF, whereas KRT14-NF signal was only visible in HaCaT cells and GAPDH-NF signal (reference signal) in both types of cells.

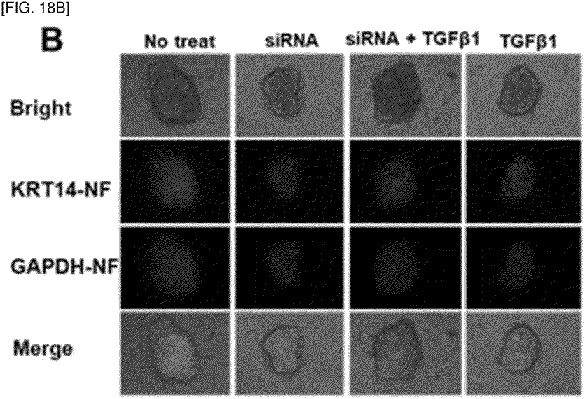

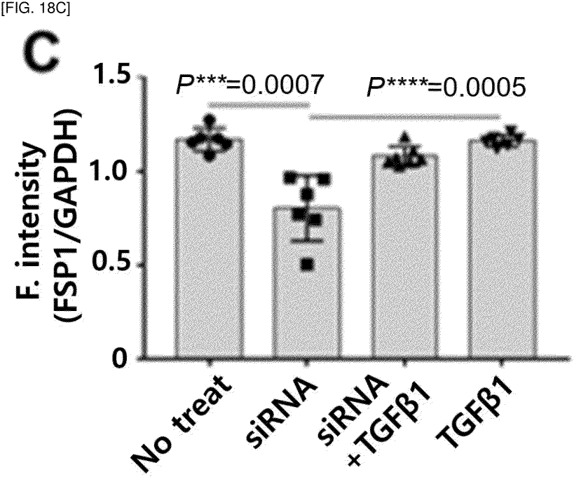

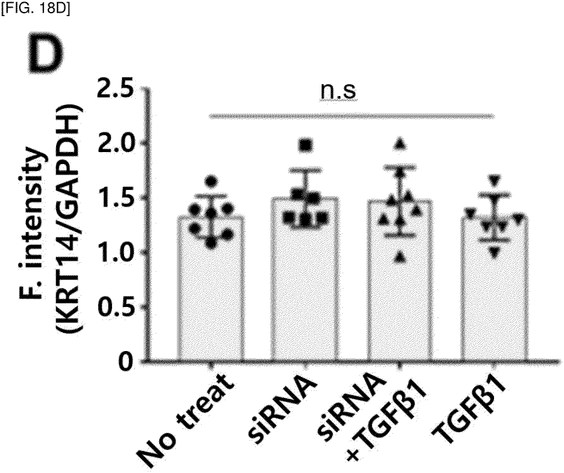

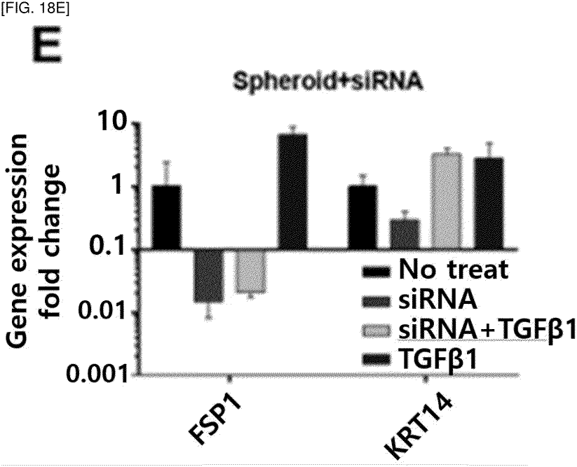

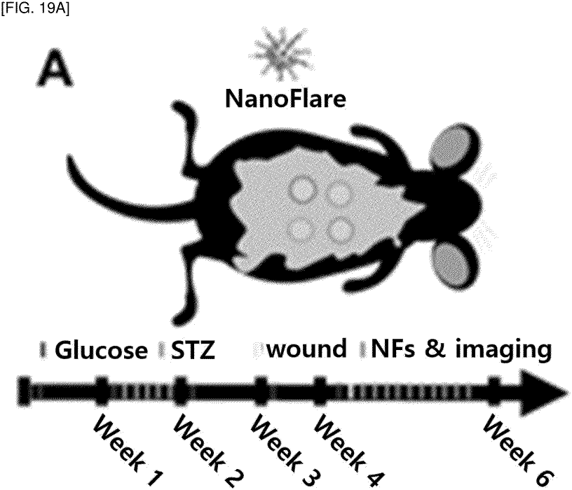

In addition, in an example embodiment of the present disclosure, examined was whether the nanoflare could detect changes in gene expression in 3D culture. As shown in FIGS. 18A to 18E, HaCaT and NDF spheroids were first treated with TGFBR1 siRNA, TGFBR1 siRNA + TGFβ1, or TGFβ1 before incubation along with KRT14-NF/GAPDH-NF and FSP1-NF/GAPDH-NF, respectively. Referring to FIGS. 18A, 18C, and 18E, in the spheroids treated with TGFBR1 siRNA and siRNA+TGFβ1, the FSP1-NF signal was decreased by 27% and 7%, respectively, but the decrease was not statistically significant in the TGFβ1-treated group. Referring to FIGS. 18B, 18D, and 18E, KRT14-NF signal remained unchanged in HaCaT spheroids under all conditions, which is identical to the qPCR results. This suggests that gene-specific nanoflares were capable of detecting changes in gene expression in 3D culture in accordance with subtle changes in TGFβ signaling and may support further development of gene-specific nanoflares in an attempt to track changes in gene expression directly in cells or tissues under physiological conditions.

To confirm the broad applicability and optimized methodology of nanoflares, the nanoflare was examined in 3D cell spheroids prepared using keratinocytes and fibroblasts from different sources, as shown in FIG. 17C. 3D cell spheroids were prepared from human (KC), human dermal papillary cells (DPC), or co-cultured KC and DPC (KCDP). KC-, DPC-, and KCDP-spheroids with dimensions of 0.01 mm³ were cultured with a mixture of KRT14-NF, FSP1-NF, and GAPDH-NF for 16 hours and then evaluated by confocal fluorescence imaging. Referring to FIG. 17C, KRT14-NF signal was specifically detected in KC- and KCDP spheroids whereas FSP1 signal was detected in DP and KCDP spheroids. In an example embodiment of the present disclosure, it was noticeable that the fluorescence signal of GAPDH-NF was detected in all cells, but the fluorescence signal of KRT14-NF and FSP1-NF did not overlap in the KCDP co-culture spheroids. All these findings suggest that nanoflares effectively penetrated labeled cells and cells within 3D spheroids with high specificity.

### <Example 6> Monitoring Wound Healing in Normal and Diabetic Mice

For the monitoring of the nanoflare signal in a normal mouse model, C57BL/6 was anesthetized with alfaxalone (30-60 mg/kg) before wound formation on the back of the mouse. Alfaxalone was diluted in PBS in a ratio of 1:10 and injected intramuscularly in the volume of 0.1 mL per mouse. Wounds were formed using a 4 mm biopsy punch, and each mouse had 4 spots. Skin was removed with care to ensure complete removal of attached connective tissues and flap exposure.

Topical applications (GAPDH-Cy5, PECAM-Cy3, KRT14-Cy3, and FSP1-Cy3) were allocated to the four wound spots of the experimental group to eliminate variability of individual wounds due to anatomical position. The sequence of topical application was clockwise. In the case of a control group, the number of wound sites was the same for the uniformity of the experiment, but only GAPDH and GNP were treated. Moisturizing cream (Aquaphor) and PBS were mixed in a ratio of 6:4 to prepare a formulation suitable for sample treatment. Each sample was then diluted in a ratio of 1:100 at a final concentration of O.D 1 and treated with 10 µL per spot. Overall, topical application was evenly distributed to each wound spot. After application of the sample, the mouse was exposed to an infrared heat lamp for 10 minutes for better absorption in the wound spot. Nanoflare samples were applied to mice daily from day 1 to day 9. After 24 hours, mice were taken for fluorescence imaging using IVIS® Spectrum CT (PerkinElmer, Singapore Pte Ltd). Field of view (16 cm) covering the entire mouse, height distance, and optical gain were remained constant throughout the measurement. Finally, the average fluorescence intensity of Cy5 and Cy3 for each region of interest was recorded.

The wound was left to heal spontaneously for 10 days. On days 2, 4, 7, and 10, 5 mice in the experimental group were euthanized. After euthanasia, the surrounding skin including the wound spots was removed and stored at -80° C. for further study.

For monitoring of nanoflare signals in a diabetic mouse model, C57BL/6 mice were anesthetized using isoflurane, and became diabetic via intraperitoneal injection of 60 mg/kg of streptozotocin (STZ, Sigma) for 5 consecutive days in citrate buffer (0.1 M sodium citrate and 0.1 M citric acid, pH 4.5). Blood glucose levels were measured by the POCT Glucose Detection Kit (GlucoCare) daily after STZ injection and for the next two consecutive weeks. Animals with a blood sugar level of 250 mg/dL or higher were considered as a diabetic group to be taken for laboratory testing. On the fourth week, mice were anesthetized using isoflurane and alfaxalone (30-60 mg/kg) prior to wound formation. Wounds were formed using the same protocol as in normal mice. Topical application of SNA and imaging were performed similarly to that in normal mice. All animal experiments were approved by the Institutional Animal Care and Use Committees (IACUC) of Korea University (KOREA-2021-0107, KOREA-2020-0198).

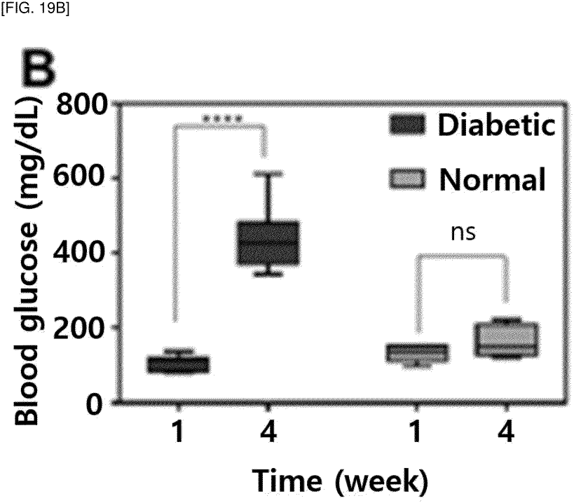

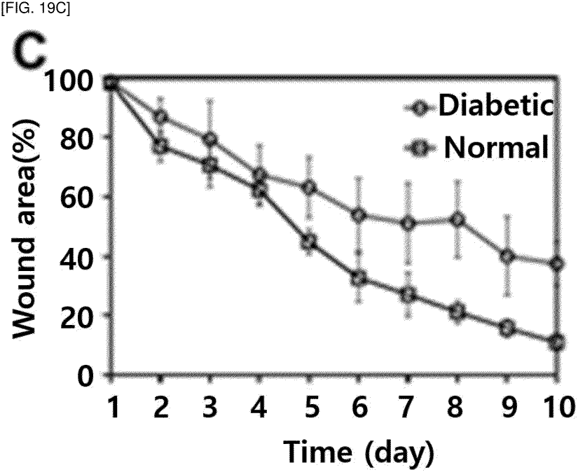

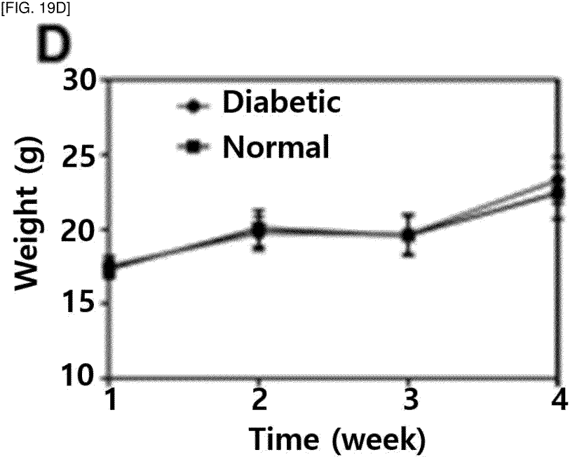

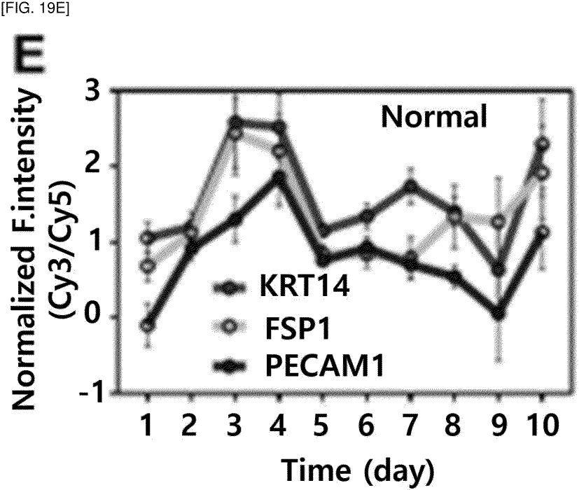

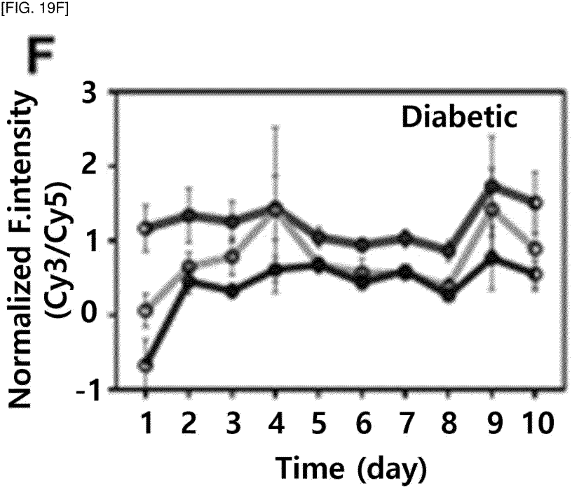

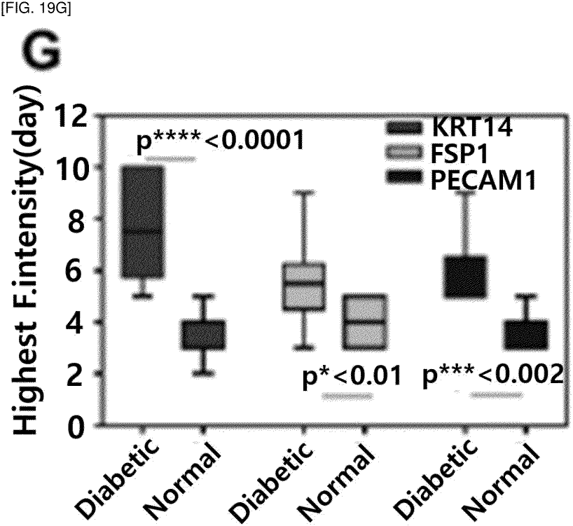

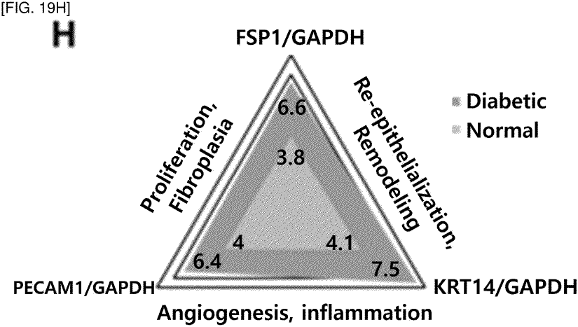

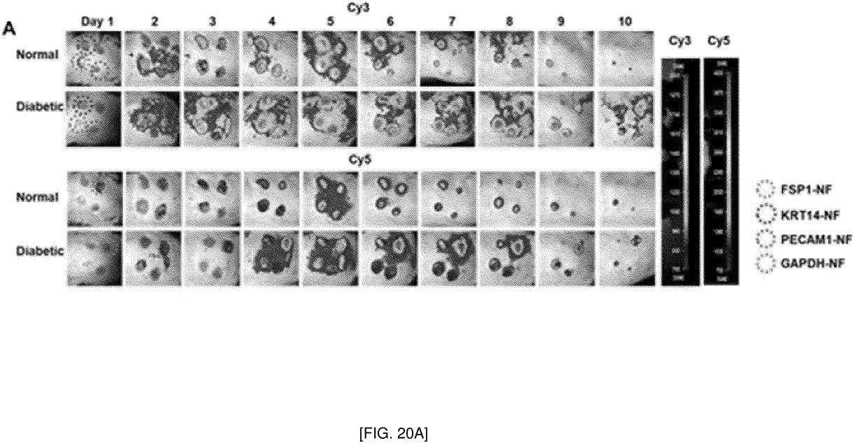

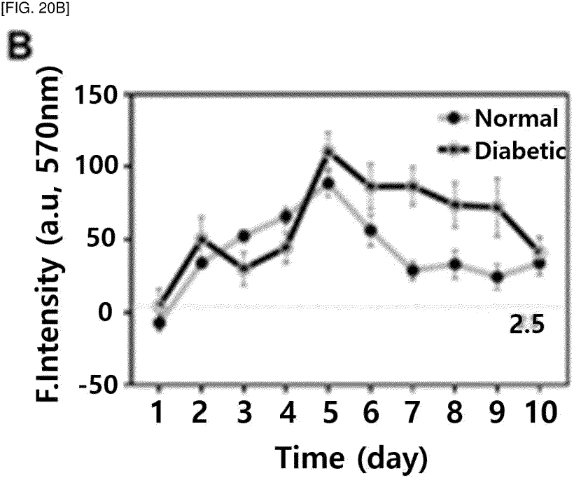

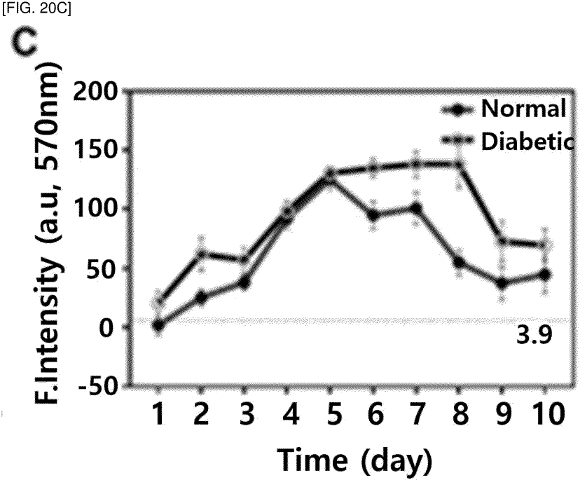

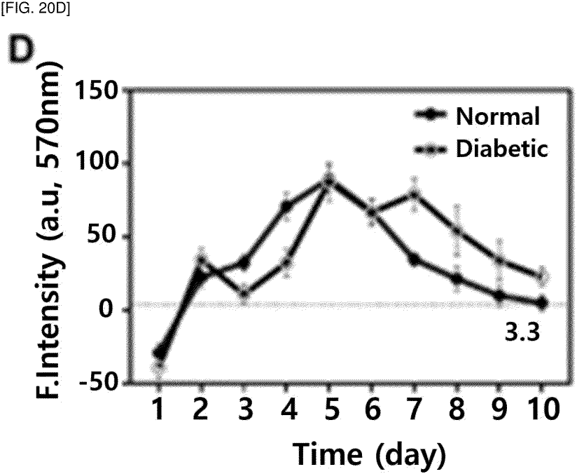

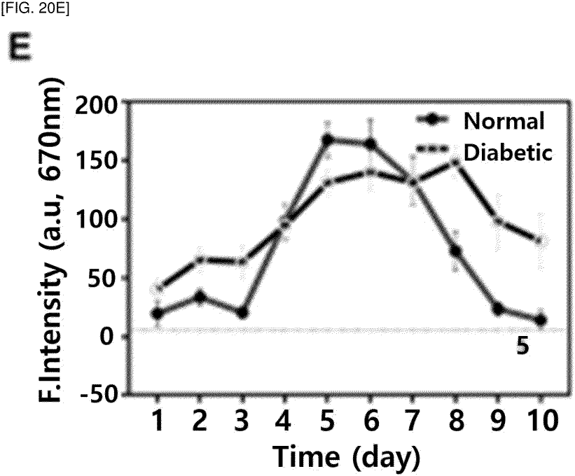

In an example embodiment of the present disclosure, it was confirmed that the designed nanoflare may target the target genes in 2D and 3D dynamic environments. Next was to determine whether the nanoflare acts similarly in vivo. In particular, in an example embodiment of the present disclosure, the nanoflare was used to study and compare wound healing in normal and diabetic mouse models. As shown in FIG. 19A, each nanoflare (GAPDH-Cy5, PECAM1-Cy3, KRT14-Cy3, and FSP1-Cy3 nanoflare) was first mixed with a moisturizer and then locally applied to the wound independently 4 hours after wound formation. As shown in FIG. 20A, in vivo fluorescence imaging was performed for 10 consecutive days afterward (the nanoflare was reapplied 1 hour after imaging from day 2 to day 9). Referring to FIG. 19B, diabetic mice showed consistently elevated blood glucose levels (440±87 mg/dL) than normal mice (107±20 mg/dL) throughout the experiment. Referring to FIG. 19C, the wound healed faster in normal mice than in diabetic mice, but referring to FIG. 19D, there was no significant difference in body weight change in the two mouse groups. The nanoflare fluorescence signal collected as in FIGS. 19E and 19F was quantified by image J, where the average fluorescence value was derived by subtracting the background signal from the fluorescence signal.

As shown in FIG. 20B, the FSP1 signal in normal wounds was observable from day 2, peaked on day 5, and diminished thereafter. Differently, FSP1 signal increased continuously at day 5 and was maintained until day 9 in diabetic wounds. The features of the KRT14 signal were similar to those of FSP1 in normal wounds, where the KRT14 signal appeared from day 2 and peaked at day 5 (FIG. 20C). However, KRT14 signal appeared on day 2 in the diabetic group and peaked on day 8. Notably, in both types of wounds, the KRT14 nanoflare signal decreased significantly slower after a peak compared to the FSP1 nanoflare signal. On the other hand, referring to FIG. 20D, the PECAM1 nanoflare signal was found from day 2 to day 7 in the normal group and reached a maximum peak on day 5. The diabetic group showed similar fluctuations, but the peak shifted to the right (occurred at a later time point). In addition, as shown in FIG. 20E, the fluorescence signal from GAPDH mRNA, a reference gene, was monitored.

FSP1, KRT14, and PECAM1 signals were normalized to the signal of GAPDH, the reference gene as shown in FIGS. 19E and 19F. Referring to FIG. 19E, in normal wounds, all three normalized signals showed similar fluctuations and peaked on days 2-4 and day 10. However, referring to FIG. 19F, there was no significant increase in diabetic wounds until day 8. Referring to 19G, there was a 2-3 day delay in the appearance of the maximum fluorescence signal.

A wound healing diagram was established as in FIG. 19H based on the fluorescence peak of each nanoflare in FIG. 19G. The fluorescence wound healing index (FWI) may be derived by comparing the maximum fluorescence of each nanoflare in the diabetic group with the fluorescence of the control group. Specifically, the maximum fluorescence signals for normal wounds were FSP1 (3.8), KRT14 (4.1), and PECAM1 (4), whereas the diabetic group showed FSP1 (6.5), KRT14 (7.5), and PECAM1 (6.4). Therefore, the FWI was 1.7 for fibrous dysplasia-proliferation (FSP1), 1.9 for re-epithelialization-remodeling (KRT14), and 1.6 for angiogenesis-inflammation (PECAM1). The values of FWI represent the deviation of diabetic wound healing from the normal range.

Taken together, the nanoflare-derived signals confirmed that diabetic wounds showed delayed wound closure, prolonged inflammation, poor angiogenesis, and less matrix deposition at the wound bed compared to normal wound healing.

### <Example 7> Determination of Degree of Wound Healing in Normal and Diabetic Mice and diabetic foot ulcers

Proposed are schematization of the wound healing process through monitoring of the affected area in diabetic foot ulcer patients as well as the probability of monitoring the wound healing process using nanosensors in vivo (animal model) thereby.

An experiment to determine the degree of wound healing using the nanoflare in a normal mouse model and a diabetic mouse model was performed as follows.

To monitor the nanoflare signal in a normal mouse model, C57BL/6 was anesthetized using alfaxalone (30-60 mg/kg) prior to formation of wound on the back of the mouse. Alfaxalone was diluted in PBS in a ratio of 1:10 and injected intramuscularly in the volume of 0.1 mL per mouse. Wounds were formed by a 4 mm biopsy punch and each mouse had 4 spots. Skin was removed with care to ensure complete removal of attached connective tissues and flap exposure.

Topical applications (GAPDH-Cy5, PECAM-Cy3, KRT14-Cy3, and FSP1-Cy3) were allocated to the four wound spots of the experimental group to eliminate variability of individual wounds due to anatomical position. The sequence of topical application was clockwise. In the case of the control group, the number of wound sites was the same for the uniformity of the experiment, but only GAPDH and GNP were treated. Moisturizing cream (Aquaphor) and PBS were mixed in a ratio of 6:4 to prepare a formulation suitable for sample treatment. Each sample was then diluted in a ratio of 1:100 to a final concentration of O.D 1 and treated with 10 µL per spot. Overall, topical application was evenly distributed to each wound spot. After application of the sample, the mouse was exposed to an infrared heat lamp for 10 minutes for better absorption in the wound spot. Nanoflare samples were applied to mice daily from day 1 to day 9. After 24 hours, mice were taken for fluorescence imaging using IVIS® Spectrum CT (PerkinElmer, Singapore Pte Ltd). Field of view (16 cm) covering the entire mouse, height distance, and optical gain remained constant throughout the measurement. Finally, the average fluorescence intensity of Cy5 and Cy3 for each region of interest was recorded.

The wound was left to heal spontaneously for 10 days.

To monitor nanoflare signals in the diabetic mouse model, C57BL/6 mice were anesthetized using isoflurane and became diabetic via intraperitoneal injection of 60 mg/kg of streptozotocin (STZ, Sigma) for 5 consecutive days in citrate buffer (0.1 M sodium citrate and 0.1 M citric acid, pH 4.5). Blood glucose levels were measured using POCT Glucose Detection Kit (GlucoCare) daily after STZ injection and for the next two consecutive weeks. Animals with blood sugar level of 250 mg/dL or higher were considered as a diabetic group and taken for laboratory testing. On the fourth week, mice were anesthetized using isoflurane and alfaxalone (30-60 mg/kg) prior to wound formation. Wounds were formed using the same protocol as in normal mice. Topical application of SNA and imaging were performed similarly to that in normal mice. All animal experiments were approved by the Institutional Animal Care and Use Committees (IACUC) of Korea University (KOREA-2021-0107, KOREA-2020-0198).

From day 1 to 10, KRT14, FSP1, PECAM1, and GAPDH nanosensors were treated on four wound sites every day respectively, and the wound sites were taken with IVIS for imaging. Then, in the imaging of each wound recovery process, an ROI (region of interest) portion (5 mm circle) was designated in the same size on the wounds in 4 areas, and a background portion (5 mm circle) around the wound site was set. The average fluorescence index was calculated by average ROI signal-average background signal of each wound site, and this was expressed as a wound healing index.

For the wound healing index by date for each wound, based on less than 2 days (fresh wound stage), less than 5 days (inflammation stage), and more than 5 days (re-epithelialization stage), the values of average fluorescence KRT14/GAPDH, FSP1/ GAPDH, and PECAM1/GAPDH for the wound site by nanoparticles were graphed by classifying the same into normal animal models (black) and diabetic models (colors).

TABLE 4

| | KRT14 NF | | FSP1 NF | | PECAM1 NF | |
|---|---|---|---|---|---|---|
| | Normal | Diabetic | Normal | Diabetic | Normal | Diabetic |
| FW | 0-1 | 0-1 | 0-1 | less than 1 | 0-1 | less than 1 |
| IS | 1.5 or more | 0-1.5 | 1-3 | 0-1 | 1-2 | less than 1 |
| PR | 1 or more | 1.5 | 1 or more | 1-1.5 | less than 1 | less than 1 |

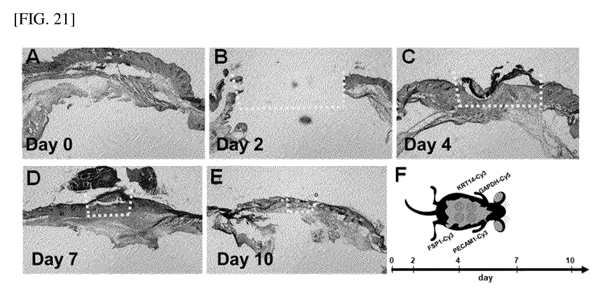

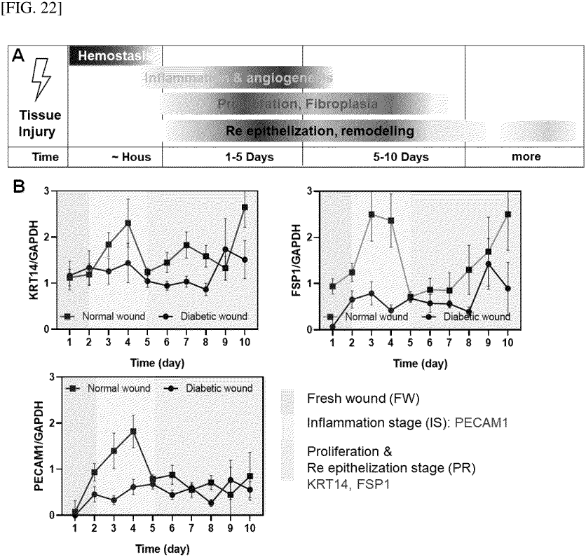

In the mouse experiment (FIG. 21F), 2 days after the wound formation (FIG. 21B), no cell formation was observed in the skin tissues. Referring to FIG. 22B, signals of the nanosensors FSP1/GAPDH, KRT14/GAPDH, and PECAM1/GAPDH each representing wound healing stage did not increase.

In addition, in the mouse experiment, formation of connective tissues, epidermal cells, and scabs was observed in the wound on day 4 after the wound formation (FIG. 21C). Referring to FIG. 22B, the signals of nanosensors FSP1/GAPDH, KRT1/GAPDH, and PECAM1/GAPDH were greatly increased, and proliferation, angiogenesis, inflammation, and reepithelization were found to be active.

Moreover, in the mouse experiment, it was observed that most of the wounds were recovered by connective tissues, epidermal cells, and scabs in the wound on day 7 after the wound formation (FIG. 21D). Referring to FIG. 22B, the nanosensors FSP1/GAPDH and KRT1/GAPDH showed a higher signal than that in the wound at an initial stage, while the signal of PECAM1/GAPDH was decreased. Thus, proliferation and reepithelization still occurred, and inflammation was found to be reduced.

In addition, in the mouse experiment, it was observed that the wound was recovered on day 10 after the wound formation (FIG. 21E). Referring to FIG. 22B, the signals of the nanosensors FSP1/GAPDH and KRT1/GAPDH decreased and then increased again. In addition, it was confirmed that the signal of PECAM1/GAPDH decreased, so that proliferation and re-epithelization occurred actively while inflammation was terminated.

The process took place in the animal experiment was shown in an image as shown in FIG. 22A.

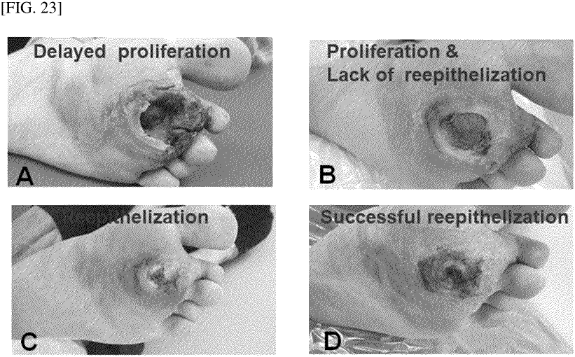

In the case of diabetic ulcers (FIGS. 23A-D ), although not imaged using the nanosensor (NF), when proliferation and re-epithalization did not seem carried out properly, deep and wide ulcers were observed, skin regeneration was observed during recovery (C), and then a scab was also observed (D).

In conclusion, in animal experiments, wound images and wound healing by nanosensors showed the same progress, and the pattern thereof was consistent even in diabetic foot ulcers. In particular, although the change in the degree of wound healing by each of the four types of nanosensors was not clear, it was distinguished from abnormal wound healing, enabling the monitoring of the wound healing process which is observable in Table 4 listed with each signal.

In addition, based on this, the stages of inflammation, proliferation, and re-epithalization were quantified and expressed as a nanosensor (fluorescence intensity of the target gene/GADPH) in normal and abnormal (diabetes) cases, thus wound healing index (WHI) was completed and shown in Table 4.

In Table 4, a fresh wound (FW) refers to a wound from day 0 to less than 2 after the wound formation, and an inflammatory stage (IS) to a wound from day 2 to less than 5 after the wound formation, and the proliferation & re-epithelialization stage (PR) to a wound that is recovered after day 5. Referring to Table 4, the wound is in the imflammation stage if an index value is 1 or more from the nanosensor including PECAM1/GAPDH, and in the proliferation & re-epithelialization stage if the index value is 1 or more respectively from the nanosensor including KRT14/GAPDH or FSP1/GAPDH.

The wound healing stage mentioned in the present disclosure is a wound healing stage produced by itself through PECAM1, FSP1, KRT14, and GAPDH, and the results of FIG. 23 were consistent with the FW (Fresh wound), IS (Imflammation stage = PECAM1), and PR (Proliferation & Re-epithelialization stage = FSP1, KRT14) parts shown in FIG. 22 and Table 4. For this reason, as in the actual clinical patient (FIG. 23), it may be used as IS (Imflammation stage = PECAM1), PR (Proliferation & Re-epithelialization stage = FSP1, KRT14), and the like, according to the wound monitoring method of each stage.

As the specific parts of the present disclosure have been described in detail above, for those of ordinary skill in the art, these specific descriptions are only preferred embodiments, and it is clear that the scope of the present disclosure is not limited thereto. Accordingly, the substantial scope of the present disclosure will be defined by the appended claims and equivalents thereof.

The scope of the present disclosure is indicated by the following claims, and all changes or modifications derived from the meaning and scope of the claims and equivalents thereof should be construed as being included in the scope of the present disclosure.

What is claimed is:

1. A nanosensor for monitoring of wound healing, comprising:
a nanoflare comprising a core part including gold nanoparticles; and a flare part comprising a recognition sequence and a flare sequence,
wherein the recognition sequence complementarily binds to a target gene,
the flare sequence complementarily binds to the recognition sequence, and
the flare part is formed on a surface of the core part.

2. The nanosensor of claim 1, wherein the target gene comprises one or more selected from the group consisting of PECAM1, FSP1, KRT14, and GAPDH.

3. The nanosensor of claim 1, wherein a base sequence recognizing the target gene is one or more of base sequences represented by SEQ ID NO: 1, SEQ ID NO: 2, SEQ ID NO: 9, SEQ ID NO: 10, SEQ ID NO: 13, SEQ ID NO: 15, and SEQ ID NO: 16.

4. The nanosensor of claim 3, wherein the base sequence recognizing the target gene comprises one of the base sequences represented by SEQ ID NO: 1, SEQ ID NO: 2, SEQ ID NO: 9, SEQ ID NO: 10, and SEQ ID NO: 13; and the base sequence represented by SEQ ID NO: 15 or SEQ ID NO: 16.

5. The nanosensor of claim 1, wherein the flare sequence consists of 14 to 18 nucleotides.

6. The nanosensor of claim 5, wherein the flare sequence comprises one or more of base sequences represented by SEQ ID NO: 3 to SEQ ID NO: 8, SEQ ID NO: 11, SEQ ID NO: 12, SEQ ID NO: 14, SEQ ID NO: 17, and SEQ ID NO: 18.

7. The nanosensor of claim 6, wherein the flare sequence comprises one base sequence represented by SEQ ID NO: 3 to SEQ ID NO: 8, SEQ ID NO: 11, SEQ ID NO: 12, or SEQ ID NO: 14; and the base sequence represented by SEQ ID NO: 17 or SEQ ID NO: 18.

8. The nanosensor of claim 1, wherein the nanosensor measures a degree of wound healing as the flare part binds to the target gene.

9. The nanosensor of claim 1, wherein the nanosensor measures a degree of wound healing as a fluorescence signal changes according to concentration of the target gene.

10. A method of manufacturing the nanosensor for monitoring of wound healing of claim 1, the method comprising:
preparing a flare part by mixing a recognition sequence and a flare sequence; and
forming a nanoflare by storing a mixture obtained by mixing the prepared flare part with gold nanoparticles at a temperature of -30 to -10° C. for 1 hour to 3 hours and reacting the mixture with a salt.

11. The method of claim 10, wherein the preparing of the flare part comprises mixing the recognition sequence and the flare sequence in a molar ratio of 1:1 to 10:1.

12. The method of claim 10, wherein the forming of the nanoflare comprises adding a salt to the mixture until the final salt concentration reaches 0.2 to 0.5 M and reacting them for 1 hour to 24 hours, and
the salt is sodium chloride.

13. The method of claim 10, wherein the forming of the nanoflare comprises mixing the prepared flare part and the gold nanoparticles in a volume ratio of 1:3 to 10.

14. A wound healing monitoring method, the method comprising:
applying the nanosensor according to claim 1 to a wound site; and
measuring a degree of wound healing through in vivo fluorescence imaging according to a fluorescent dye included in the flare sequence as the recognition sequence in the applied nanosensor binds with a target gene in the wound site.

15. The method of claim 14, wherein the applying of the nanosensor to the wound site comprises applying a mixture of an emulsion solution and the nanosensor.

16. The method of claim 14, wherein the measuring of the degree of wound healing comprises measuring an intensity of fluorescence of the fluorescent dye included in the flare sequence.

17. The method of claim 16, wherein, in the measuring of the degree of wound healing, the degree of wound healing is an inflammatory reaction stage when a wound healing index of a PECAM1/GAPDH nanosensor is 1 or more after two days.

18. The method of claim 16, wherein, in the measuring of the degree of wound healing, the degree of wound healing is a proliferation & re-epithelialization stage when a wound healing index of a PECAM1/GAPDH nanosensor is less than 1 after five days.

19. The method of claim 16, wherein, in the measuring of the degree of wound healing, the degree of wound healing is a proliferation & re-epithelialization stage when a wound healing index of at least one of KRT14/GAPDH and FSP1/GAPDH nanosensors is 1 or more after five days.

20. The method of claim 16, wherein, in the measuring of the degree of wound healing, the degree of wound healing is a proliferation & re-epithelialization stage when a wound healing index of at least one of KRT14/GAPDH and FSP1/GAPDH nanosensors is 1 or more, and the wound healing index of a PECAM1/GAPDH nanosensor is less than 1 after seven days.


SEQUENCE LISTING (90 sequences)

SEQ ID NO: 1 - DNA, 34 bp, synthetic construct
  source 1..34: mol_type = other DNA
atttcttcctgggctgcttatctgggaaaaaaaa

SEQ ID NO: 2 - DNA, 34 bp, synthetic construct
  source 1..34: mol_type = other DNA
acttcttccggggctccttatctgggcaaaaaaa

SEQ ID NO: 3 - DNA, 18 bp, synthetic construct
  source 1..18: mol_type = other DNA
cccgacgaatagaccctt

SEQ ID NO: 4 - DNA, 16 bp, synthetic construct
  source 1..16: mol_type = other DNA
cgacgaatagaccctt

SEQ ID NO: 5 - DNA, 15 bp, synthetic construct
  source 1..15: mol_type = other DNA
gacgaatagaccctt

SEQ ID NO: 6 - DNA, 15 bp, synthetic construct
  source 1..15: mol_type = other DNA
gaggaatagacccgt

SEQ ID NO: 7 - DNA, 14 bp, synthetic construct
  source 1..14: mol_type = other DNA
acgaatagaccctt

SEQ ID NO: 8 - DNA, 12 bp, synthetic construct
  source 1..12: mol_type = other DNA
gaatagaccctt

SEQ ID NO: 9 - DNA, 34 bp, synthetic construct
  source 1..34: mol_type = other DNA
atggagcaggacaggttcagtctttcacaaaaaa

SEQ ID NO: 10 - DNA, 36 bp, synthetic construct
  source 1..36: mol_type = other DNA
ttaagggagccttccgttcttagggtcgacaaaaaa

SEQ ID NO: 11 - DNA, 15 bp, synthetic construct
  source 1..15: mol_type = other DNA
ccaagtcagaaagtg

SEQ ID NO: 12 - DNA, 15 bp, synthetic construct
  source 1..15: mol_type = other DNA
caagaatcccagctg

SEQ ID NO: 13 - DNA, 34 bp, synthetic construct
  source 1..34: mol_type = other DNA
gactgcagctcaatctccaggttctgcaaaaaaa

SEQ ID NO: 14 - DNA, 15 bp, synthetic construct
  source 1..15: mol_type = other DNA
agaggtccaagacgt

SEQ ID NO: 15 - DNA, 34 bp, synthetic construct
  source 1..34: mol_type = other DNA
gatggcatggactgtggtcatgagtcctaaaaaa

SEQ ID NO: 16 - DNA, 34 bp, synthetic construct
  source 1..34: mol_type = other DNA
gatggcatggactgtggtcatgagccctaaaaaa

SEQ ID NO: 17 - DNA, 15 bp, synthetic construct
  source 1..15: mol_type = other DNA
caccagtactcagga

SEQ ID NO: 18 - DNA, 15 bp, synthetic construct
  source 1..15: mol_type = other DNA
caccagtactcggga

SEQ ID NO: 19 - DNA, 21 bp, synthetic construct
  source 1..21: mol_type = other DNA
tgagcaacttggacagcaaca

SEQ ID NO: 20 - DNA, 23 bp, synthetic construct
  source 1..23: mol_type = other DNA
atttcttcctgggctgcttatct

SEQ ID NO: 21 - DNA, 19 bp, synthetic construct
  source 1..19: mol_type = other DNA
cagcatggacgttcgtctg

SEQ ID NO: 22 - DNA, 20 bp, synthetic construct
  source 1..20: mol_type = other DNA
aaccacggtttggtccttgg

SEQ ID NO: 23 - DNA, 20 bp, synthetic construct
  source 1..20: mol_type = other DNA
tctgcgacaacggcaaggtg

SEQ ID NO: 24 - DNA, 20 bp, synthetic construct
  source 1..20: mol_type = other DNA
gacgccggtggtttcttggt

SEQ ID NO: 25 - DNA, 22 bp, synthetic construct
  source 1..22: mol_type = other DNA
aaccgtggcttcatggtgactc

SEQ ID NO: 26 - DNA, 22 bp, synthetic construct
  source 1..22: mol_type = other DNA
tgatgaggctggagagcttgtg

SEQ ID NO: 27 - DNA, 22 bp, synthetic construct
  source 1..22: mol_type = other DNA
ctatgcctctggacgcacaact

SEQ ID NO: 28 - DNA, 22 bp, synthetic construct
  source 1..22: mol_type = other DNA
cagatccagacgcatgatggca

SEQ ID NO: 29 - DNA, 24 bp, synthetic construct
  source 1..24: mol_type = other DNA
cacgcagatttattccagtgaaac

SEQ ID NO: 30 - DNA, 22 bp, synthetic construct
  source 1..22: mol_type = other DNA
gaccacgaagagactggctgtt

SEQ ID NO: 31 - DNA, 22 bp, synthetic construct
  source 1..22: mol_type = other DNA
aaaggcacctgggaagtcatcg

SEQ ID NO: 32 - DNA, 22 bp, synthetic construct
  source 1..22: mol_type = other DNA
cagctcacaactgaggcatgtc

SEQ ID NO: 33 - DNA, 22 bp, synthetic construct
  source 1..22: mol_type = other DNA
gaaggtcctctacttatccgcc

SEQ ID NO: 34 - DNA, 22 bp, synthetic construct
  source 1..22: mol_type = other DNA
tgatgccctcacacttgaccag

SEQ ID NO: 35 - DNA, 22 bp, synthetic construct
  source 1..22: mol_type = other DNA
agatcgactccagcaccgtaga

SEQ ID NO: 36 - DNA, 24 bp, synthetic construct
  source 1..24: mol_type = other DNA
ctctaccaaaacttcaacgctgac

SEQ ID NO: 37 - DNA, 22 bp, synthetic construct
  source 1..22: mol_type = other DNA
aacgagagtgccaccaatggct

SEQ ID NO: 38 - DNA, 22 bp, synthetic construct
  source 1..22: mol_type = other DNA
actcactggcttcagagcggaa

SEQ ID NO: 39 - DNA, 22 bp, synthetic construct
  source 1..22: mol_type = other DNA
agcgagtggatgccgcctttaa

SEQ ID NO: 40 - DNA, 22 bp, synthetic construct
  source 1..22: mol_type = other DNA
cattccaggcatctgcgatgag

SEQ ID NO: 41 - DNA, 22 bp, synthetic construct
  source 1..22: mol_type = other DNA
aggcaaagcaggagtccactga

SEQ ID NO: 42 - DNA, 22 bp, synthetic construct
  source 1..22: mol_type = other DNA
atctggcgttccagggactcat

SEQ ID NO: 43 - DNA, 22 bp, synthetic construct
  source 1..22: mol_type = other DNA
cctgcttcagatcgacaatgcc

SEQ ID NO: 44 - DNA, 22 bp, synthetic construct
  source 1..22: mol_type = other DNA
atctccaggtcagccttggtca

SEQ ID NO: 45 - DNA, 22 bp, synthetic construct
  source 1..22: mol_type = other DNA
tgccgaggaatggttcttcacc

SEQ ID NO: 46 - DNA, 22 bp, synthetic construct
  source 1..22: mol_type = other DNA
gcagctcaatctccaggttctg

SEQ ID NO: 47 - DNA, 23 bp, synthetic construct
  source 1..23: mol_type = other DNA
cagcatcattgctgaggtcaagg

SEQ ID NO: 48 - DNA, 22 bp, synthetic construct
  source 1..22: mol_type = other DNA
catgtctgccagcagtgatctg

SEQ ID NO: 49 - DNA, 22 bp, synthetic construct
  source 1..22: mol_type = other DNA
gctgcctacatgaacaaggtgg

SEQ ID NO: 50 - DNA, 22 bp, synthetic construct
  source 1..22: mol_type = other DNA
atggagaggaccactgaggtgt

SEQ ID NO: 51 - DNA, 22 bp, synthetic construct
  source 1..22: mol_type = other DNA
ctacctgaggaagaaccacgag

SEQ ID NO: 52 - DNA, 22 bp, synthetic construct
  source 1..22: mol_type = other DNA
ctcgtactggtcacgcatctca

SEQ ID NO: 53 - DNA, 22 bp, synthetic construct
  source 1..22: mol_type = other DNA
ggtccaagacattcaaccagcc

SEQ ID NO: 54 - DNA, 22 bp, synthetic construct
  source 1..22: mol_type = other DNA
tctggacactgcgggtggttat

SEQ ID NO: 55 - DNA, 23 bp, synthetic construct
  source 1..23: mol_type = other DNA
gctgaaggaacttctggaaaagg

SEQ ID NO: 56 - DNA, 24 bp, synthetic construct
  source 1..24: mol_type = other DNA
gttgtggtctatatccaagtgatc

SEQ ID NO: 57 - DNA, 22 bp, synthetic construct
  source 1..22: mol_type = other DNA
gaacgactgctggatgaagagg

SEQ ID NO: 58 - DNA, 22 bp, synthetic construct
  source 1..22: mol_type = other DNA
cttgatggactccacagagcag

SEQ ID NO: 59 - DNA, 22 bp, synthetic construct
  source 1..22: mol_type = other DNA
cgactaggagacatcttccgag

SEQ ID NO: 60 - DNA, 22 bp, synthetic construct
  source 1..22: mol_type = other DNA
gcagtgatggaaccaacggtct

SEQ ID NO: 61 - DNA, 22 bp, synthetic construct
  source 1..22: mol_type = other DNA
cgaggacttcaagtacgtgctg

SEQ ID NO: 62 - DNA, 22 bp, synthetic construct
  source 1..22: mol_type = other DNA
gtggacgcattccaggtcttga

SEQ ID NO: 63 - DNA, 22 bp, synthetic construct
  source 1..22: mol_type = other DNA
ggtggatgtgttcacgaagagg

SEQ ID NO: 64 - DNA, 23 bp, synthetic construct
  source 1..23: mol_type = other DNA
ccttcttgaaccagctctgcttc

SEQ ID NO: 65 - DNA, 21 bp, synthetic construct
  source 1..21: mol_type = other DNA
cagtgccagcagaaatatcct

SEQ ID NO: 66 - DNA, 24 bp, synthetic construct
  source 1..24: mol_type = other DNA
ccaaatatccttatcctttcttgg

SEQ ID NO: 67 - DNA, 22 bp, synthetic construct
  source 1..22: mol_type = other DNA
aagtggagtccagccgcatatc

SEQ ID NO: 68 - DNA, 22 bp, synthetic construct
  source 1..22: mol_type = other DNA
atggagcaggacaggttcagtc

SEQ ID NO: 69 - DNA, 22 bp, synthetic construct
  source 1..22: mol_type = other DNA
ggaacctcactatccgcagagt

SEQ ID NO: 70 - DNA, 22 bp, synthetic construct
  source 1..22: mol_type = other DNA
ccaagttcgtcttttcctgggc

SEQ ID NO: 71 - DNA, 23 bp, synthetic construct
  source 1..23: mol_type = other DNA
cctgcaagattcaggcacctatg

SEQ ID NO: 72 - DNA, 21 bp, synthetic construct
  source 1..21: mol_type = other DNA
ttgcagtgctcacctctgatt

SEQ ID NO: 73 - DNA, 22 bp, synthetic construct
  source 1..22: mol_type = other DNA
cctcagtgtctactgctggtct

SEQ ID NO: 74 - DNA, 22 bp, synthetic construct
  source 1..22: mol_type = other DNA
ggaatagctctggtggcttgca

SEQ ID NO: 75 - DNA, 22 bp, synthetic construct
  source 1..22: mol_type = other DNA
cggtggtcaatatcctgtcgag

SEQ ID NO: 76 - DNA, 22 bp, synthetic construct
  source 1..22: mol_type = other DNA
aggaagtgtgggctgaggtaga

SEQ ID NO: 77 - DNA, 22 bp, synthetic construct
  source 1..22: mol_type = other DNA
atcgctgctgagtgaaccacag

SEQ ID NO: 78 - DNA, 22 bp, synthetic construct
  source 1..22: mol_type = other DNA
ctactctctgcctcacaggtca

SEQ ID NO: 79 - DNA, 22 bp, synthetic construct
  source 1..22: mol_type = other DNA
agcggctgacgtgtgcagtaat

SEQ ID NO: 80 - DNA, 22 bp, synthetic construct
  source 1..22: mol_type = other DNA
tctgagacctctggcttcgtca

SEQ ID NO: 81 - DNA, 22 bp, synthetic construct
  source 1..22: mol_type = other DNA
gattctgtgcccacagtaaggc

SEQ ID NO: 82 - DNA, 22 bp, synthetic construct
  source 1..22: mol_type = other DNA
tggtcacagagccaccttcttg

SEQ ID NO: 83 - DNA, 22 bp, synthetic construct
  source 1..22: mol_type = other DNA
gaagcctctgattggcacagtg

SEQ ID NO: 84 - DNA, 23 bp, synthetic construct
  source 1..23: mol_type = other DNA
ttttgtgactcggaagaactggc

SEQ ID NO: 85 - DNA, 22 bp, synthetic construct
  source 1..22: mol_type = other DNA
ccttgaatcccagtgaccctga

SEQ ID NO: 86 - DNA, 22 bp, synthetic construct
  source 1..22: mol_type = other DNA
ggttccgagatgtcctccacat

SEQ ID NO: 87 - DNA, 22 bp, synthetic construct
  source 1..22: mol_type = other DNA
ggtcaagcaacccagccttttc

SEQ ID NO: 88 - DNA, 22 bp, synthetic construct
  source 1..22: mol_type = other DNA
caggtcattccagcagagccaa

SEQ ID NO: 89 - DNA, 22 bp, synthetic construct
  source 1..22: mol_type = other DNA
cctgtgacatcctcttcagagc

SEQ ID NO: 90 - DNA, 22 bp, synthetic construct
  source 1..22: mol_type = other DNA
cacttgcagcaggacatgatcc